SEQUENCE LISTING (8 sequences)

SEQ ID NO: 1 - DNA, 2035 bp, Artificial Sequence
  nucleotide sequence encoding PAP I cloned to pCGN1559
acttataccttttacaatttgtttatatattttacgtatctatctttgttccatggagggttcgtccaaagggctgcgaaaaggtgcttggactactgaagaagatagtctcttgagacagtgcattaataagtatggagaaggcaaatggcaccaagttcctgtaagagctggtatgttatttacgaacacacacacactaaccgacacacacacacacaaatatgaatatctataatcactaccaatagtcttcgttctctctattttctattcagaaaattgattaatacccggtattaaaaaaaaaaaaaaaaatttgtttaaatgagtacaaatcattgttacaacttctttatgctgtttttacatgctattaaaggttgtgcatgaaaatttcttttgctgttcgtatttgttttacacctaaacgaagatttttacttaaaattaaagaaaaaaaattatactaattttagttacgttgcgtattgctagcttctcctataaagtcgttcaaatttttacacgcttgtcttcttgtaaatgaattcgtgggaaaattttgtatgaacacgtgtttctgtgttggaacagttctttatttttattggtgtgcatagattcttcctgataaaatatatagaaggagacaaataaaaaacagtcttagtatgtaggtataatcaaagaatcaattattggttttgtagggctaaaccggtgcaggaaaagttgtagattaagatggttgaactatttgaagccaagtatcaagagaggaaaacttagctctgatgaagtcgatcttcttcttcgccttcataggcttctagggaataggtattaattgttacctcgatactacttaactcggagagtcgtcataagttaatactaataacatatgtatattttcttacaattgttaggtggtctttaattgctggaagattacctggtcggaccgcaaatgacgtcaagaattactggaacactcatctgagtaagaaacatgaaccgtgttgtaagataaagatgaaaaagagagacattacgcccattcctacaacaccggcactaaaaaacaatgtttataagcctcgacctcgatccttcacagttaacaacgactgcaaccatctcaatgccccaccaaaagttgacgttaatcctccatgccttggacttaacatcaataatgtttgtgacaatagtatcatatacaacaaagataagaagaaagaccaactagtgaataatttgattgatggagataatatgtggttagagaaattcctagaggaaagccaagaggtagatattttggttcctgaagcgacgacaacagaaaagggggacaccttggcttttgacgttgatcaactttggagtcttttcgatggagagactgtgaaatttgattagtgtttcgaacatttgtttgcgtttgtgtataggtttgctttcaccttttaatttgtgtgttttgataaataagctaatagtttttagcattttaatgaaatatttcaagtttccgtgtttacattttgaagaaaataaaatattaatatattctgaagatttttgtttttttttggttatctacatgacaacagtaaaaatagaaaaaaaatcttattttttgaaaaaggtatgtatccggtgtttagaatactttccgaaatcaaaccgcctatatttctaatcactatgtaaaattgtaaaccaattgggttaaaactcaactaacaaactttctaaataaatgtcatttttgttttcaaatatgattgaactcggatttaggagttttacccttcagtaccaaaccttctctaccgaccatgtatggttgggcaaatgtcatgttttacaatgtttagattactaaacactttggttgagaaggcaatgctttatttatatattctgaagtcatgttttagtgttatttttatttatttttaaatgcatagattgttaacgtgcagattctcatatgggcttagtttctggattttgactgcag

SEQ ID NO: 2 - protein, 248 aa, Artificial Sequence
  Amino Acid sequence encoding the PAP1 Protein
MEGSSKGLRKGAWTTEEDSLLRQCINKYGEGKWHQVPVRAGLNRCRKSCRLRWLNYLKPSIKRGKLSSDEVDLLLRLHRLLGNRWSLIAGRLPGRTANDVKNYWNTHLSKKHEPCCKIKMKKRDITPIPTTPALKNNVYKPRPRSFTVNNDCNHLNAPPKVDVNPPCLGLNINNVCDNSIIYNKDKKKDQLVNNLIDGDNMWLEKFLEESQEVDILVPEATTTEKGDTLAFDVDQLWSLFDGETVKFD

SEQ ID NO: 3 - DNA, 788 bp, Agrobacterium tumefaciens
cctgctttaatgagatatgcgagacgcctatgatcgcatgatatttgctttcaattctgttgtgcacgttgtaaaaaacctgagcatgtgtagctcagatccttaccgccggtttcggttcattctaatgaatatatcacccgttactatcgtatttttatgaataatattctccgttcaatttactgattgtaccctactacttatatgtacaatattaaaatgaaaacaatatattgtgctgaataggtttatagcgacatctatgatagagcgccacaataacaaacaattgcgttttattattacaaatccaattttaaaaaaagcggcagaaccggtcaaacctaaaagactgattacataaatcttattcaaatttcaaaagtgccccaggggctagtatctacgacacaccgagcggcgaactaataacgctcactgaagggaactccggttccccgccggcgcgcatgggtgagattccttgaagttgagtattggccgtccgctctaccgaaagttacgggcaccattcaacccggtccagcacggcggcaaatgaagtgcaggtcaaaccttgacagtgacgacaaatcgttgggcgggtccagggcgaattttgcgacaacatgtcgaggctcagcaggggctcgatcccctcgcgagttggttcagctgctgcctgaggctggacgacctcgcggagttctaccggcagtgcaaatccgtcggcatccaggaaaccagcagcggctatccgcgcatccatgcccccgaactgcagg

SEQ ID NO: 4 - DNA, 362 bp, Cauliflower mosaic virus
ctcagaagaccagagggctattgagacttttcaacaaagggtaatatcgggaaacctcctcggattccattgcccagctatctgtcacttcatcgaaaggacagtagaaaaggaaggtggctcctacaaatgccatcattgcgataaaggaaaggctatcgttcaagatgcctctaccgacagtggtcccaaagatggacccccacccacgaggaacatcgtggaaaaagaagacgttccaaccacgtcttcaaagcaagtggattgatgtgatatctccactgacgtaagggatgacgcacaatcccactatccttcgcaagacccttcctctatataaggaagttcatttcatttggaga

SEQ ID NO: 5 - DNA, 21 bp, Artificial Sequence
  Single strand DNA oligonucleotide
ttcctacaacaccggcactaa

SEQ ID NO: 6 - DNA, 20 bp, Artificial Sequence
  Single strand DNA oligonucleotide
tttctgttgtcgtcgcttca

SEQ ID NO: 7 - DNA, 19 bp, Artificial Sequence
  Single strand DNA oligonucleotide
acgcccattcctacaacac

SEQ ID NO: 8 - DNA, 21 bp, Artificial Sequence
  Single strand DNA oligonucleotide
tctctccatcgaaaagactcc

(19) United States
(12) Patent Application Publication
(10) Pub. No.: US 2017/0367282 A1
(43) Pub. Date: Dec. 28, 2017

(54) GYPSOPHILA PANICULATA PLANT COMPRISING A FLOWER PRODUCING COLOR PIGMENTATION

(71) Applicant: Imaginature Ltd., Moshav Mishmar HaShiva (IL)

(72) Inventors: Hadas PRICE, Moshav Givat Yeshayahu (IL); Amir ZUKER, Nes Ziona (IL); Alexander VAINSTEIN, Rechovot (IL); Gavriel DANZIGER, Moshav Nir-Zvi (IL)

(21) Appl. No.: 15/538,243

(22) PCT Filed: Dec. 23, 2015
(86) PCT No.: PCT/IL2015/051251

§ 371 Date: Jun. 21, 2017

Related U.S. Application Data
(60) Provisional application No. 62/096,039, filed on Dec. 23, 2014.

(51) Int. Cl. A01H 5/02; C12N 9/16; A01H 1/04; C12N 15/82; A01H 1/02

(52) U.S. Cl. CPC A01H 5/0227; C12Y 301/03004; C12N 15/825; A01H 1/02; A01H 1/04; C12N 9/16

(57) ABSTRACT

A Gypsophila paniculata plant comprising an exogenous nucleic acid sequence encoding PAP1 is provided. Alternatively or additionally there is provided a Gypsophila paniculata plant comprising a flower producing a non-thermally induced red, pink, purple or green pigmentation or a combination of same.

### FIELD AND BACKGROUND OF THE INVENTION

The present invention, in some embodiments thereof, relates to Gypsophila paniculata plants.

Color is one of the most important traits in the flower industry. Flower color is the most important trait, yet stems and leaves color, patterns and shades; all contribute to the quality and desirability of commercial cut flower, garden and pot plants. The color in flowers, stems, leaves and other aerial parts of the plant derived from production of two main groups of pigment groups: carotenoids and anthocyanins. Biologically, carotenoids accumulate in the chromoplast, while anthocyanins accumulate in the vacuole. Anthocyanins, change their color with acidity and thus produce a wide range of colors and shades, which range from yellow through blue and red to deep purple. Anthocyanin production in plant cells is orchestrated by a large number of genes, coding for large network of enzymes. The complexity of the Anthocyanin-production pathways in different plant species and the lack of knowledge on their generic markup of commercially important ornamental plant species, hinder our ability to rationally breed and control the production of anthocyanins in the flowers, stems, leaves and other aerial parts of ornamental plant species.

Plants of the Caryophyllaceae, among them the genus Gypsophila, are known to contain anthocyanins as their flower pigments. The most common commercial variety G. paniculata has predominantly white flowers and in rare cases there are varieties with light pink to pink flowers, like My Pink, which is characterized by color stability that depends on environmental conditions. Of the many species of Gypsophila. G. paniculata is the only one used as a cut flower, and as such it is among the most important flower crops worldwide. This makes Gypsophila an important target for the breeding of new varieties with novel characteristics. Since flowers of commercial varieties are usually sterile, directed breeding of Gypsophila plants with novel horticultural traits in general, and flower color, in particular is rather tedious and somewhat impossible.

### ADDITIONAL BACKGROUND ART INCLUDES

www(dot)aphis(dot)usda(dot)gov/biotechnology/downloads/reg_loi/Danziger_USDA_012012(dot)pdf
departments(dot)agri(dot)huji(dot)ac(dot)il/plantscience/people/Alexander_Vainstein/
U.S. Patent Application Publication No. 2003/0088888;
Moyal Ben Zvi. M., Zuker, A., Ovadis, M., Shklarman. E., Ben-Meir, H., Zenvirt, S. and Vainstein, A. (0.2008) Agrobacterium-mediated transformation of gypsophila (Gypsophila paniculata L.) Mol. Breeding 22:543-553.
Zuker A, Ahroni A, Shejtman H, Vainstein A (1997). Adventitious shoot regeneration from leaf explants of Gypsophila paniculata L. Plant Cell Rep. 16, 775-778.
Mintz-Oron, S., Mandel. T., Rogachev. I., Feldberg. L., Lotan, O., Yativ, M., Wang. Z., Jetter, R., Venger, I., Adato, A., and Aharoni, A. (2008). Gene expression and metabolism in tomato fruit surface tissues. Plant Physiol 147, 823-851.

### SUMMARY OF THE INVENTION

According to an aspect of some embodiments of the present invention there is provided a Gypsophila paniculata plant comprising an exogenous nucleic acid sequence encoding PAP1.

According to an aspect of some embodiments of the present invention there is provided a Gypsophila paniculata plant comprising a flower producing a non-thermally induced red, pink, purple or green pigmentation or a combination of same.

According to some embodiments of the invention, the plant comprises an exogenous nucleic acid sequence encoding PAP1.

According to some embodiments of the invention, the exogenous nucleic acid sequence encoding PAP1 is stably integrated in a genome of the plant.

According to some embodiments of the invention, the exogenous nucleic acid sequence encoding PAP1 is comprised in multiple copies within said genome.

According to some embodiments of the invention, the plant is a polyploid.

According to some embodiments of the invention, the plant is a tetraploid.

According to some embodiments of the invention, a pedigree which includes a Million Stars™ polyploid.

According to some embodiments of the invention, the plant has a pedigree which includes Million Stars™.

According to some embodiments of the invention, the plant is a transgenic plant.

According to some embodiments of the invention, the PAP1 comprises an amino acid sequence as set forth in SEQ ID NO: 2.

According to some embodiments of the invention, the plant has flower petals that contain cyanidin as the major anthocyanin.

According to some embodiments of the invention, the flower petals further comprise peonidin, and pelargonidin derivates.

According to some embodiments of the invention, the cyanidin comprises cyanidin malylglucoside and cyanidin hexose.

According to some embodiments of the invention, the cyanidin malylglucoside and the cyanidin hexose are about 80-90% and 10-20% respectively, of total anthocyanin content of the flower petals, as assayed by UPLC-QTOF-MS.

According to some embodiments of the invention, the plant has flower petals that contain at least one of:

(i) at least 10, 20, 50, 100 or 150 fold increase in cyanidin malylglucoside than that found in My Pink™ at the same developmental stage and assay conditions;

(ii) at least 1,000, 2,000, 3000, 5,000, 8,000 or 10,000 fold increase in cyanidin malylglucoside than that found in Million Stars™ at the same developmental stage and assay conditions;

(iii) at least 10, 20, 50, 100 150 fold increase in cyanidin hexose than that found in My Pink™ at the same developmental stage and assay conditions;

(iv) at least 100, 200, 300, 500 or 1000 fold increase in peonidin coumaroyl pentose than that found in My Pink™ at the same developmental stage and assay conditions;

(v) at least 50, 100, 200 or 500 fold increase in cyanidin pentose deoxyhexose than that found in My Pink™ at the same developmental stage and assay conditions;

According to an aspect of some embodiments of the present invention there is provided a part of the plant.

According to some embodiments of the invention, the part of the plant is selected from the group consisting of leaf, pollen, embryo, cotyledon, hypocotyls, mertistem, root, root tip, pistil, anther, flower, stem, ovule, seed and petiole.

According to some embodiments of the invention, the plant part is selected from the group consisting of a leaf, anther, stem, sepal and pistil and wherein the plant part exhibits an cyanidin level higher than that found in Gypsophyla paniculata var. Million Starts™ being of the same developmental stage and growth conditions.

According to an aspect of some embodiments of the present invention there is provided a flower of the plant.

According to some embodiments of the invention, the flower is a cut-flower.

According to an aspect of some embodiments of the present invention there is provided a pollen of the plant.

According to an aspect of some embodiments of the present invention there is provided a seed of the plant.

According to an aspect of some embodiments of the present invention there is provided an ovule of the plant.

According to an aspect of some embodiments of the present invention there is provided a cutting of the plant.

According to an aspect of some embodiments of the present invention there is provided a tissue culture comprising cells of the plant.

According to some embodiments of the invention, the plant is a hybrid plant.

According to some embodiments of the invention, the plant is an inbred plant.

According to some embodiments of the invention, the hybrid plant or inbred plant is polyploid.

According to an aspect of some embodiments of the present invention there is provided a method of producing a Gypsophila paniculata plant, the method comprising:

(a) crossing the plant or plant part with another Gypsophila plant e.g., Gypsophila paniculata plant;
(b) recovering seeds following the crossing;
(c) planting the seeds and growing the seed into plants; and
(d) selecting a hybrid plant.

According to some embodiments of the invention, the selecting is according to pigmentation.

According to an aspect of some embodiments of the present invention there is provided a hybrid plant or part thereof produced according to the method.

According to an aspect of some embodiments of the present invention there is provided a method of developing a cultivated plant using plant breeding techniques, the method comprising using the plant or plant part as a source of breeding material for self-breeding and/or cross-breeding.

According to an aspect of some embodiments of the present invention there is provided a method of developing the plant, the method comprising:

(a) crossing a Gypsohila paniculata plant with another Gypsohila paniculata plant so at to obtain hybrid seeds;

(b) growing plants of the hybrid seeds; and

(c) selecting a plant of the plants that exhibits a flower producing a non-thermally induced red, pink, purple or green pigmentation or a combination of same.

According to an aspect of some embodiments of the present invention there is provided a method of producing a transgenic Gypsophila paniculata comprising introducing into a Gypsophila paniculata plant a nucleic acid sequence encoding PAP1 operably linked to a cis-acting regulatory element active in a plant cell, thereby producing a transgenic Gypsophila paniculata.

According to some embodiments of the invention, the method further comprises subjecting the Gypsophila paniculata plant to polyploidization protocol.

According to some embodiments of the invention, the Gypsophila paniculata plant has a Million Starts™ polyploid genetic background.

According to some embodiments of the invention, the PAP1 is as set forth in SEQ ID NO: 2 or a homolog of the SEQ ID NO: 2.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE SEVERAL VIEWS OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a graph showing the DNA content of cells taken from leaves of Million Stars™ (M.S.).

FIG. 2 is a graph showing the DNA content of cells taken from leaves of M.S polyploid.

FIG. 3 is a schematic illustration of the Arabidopsis pap I gene (SEQ ID NO: 1) cloned to pHAPAP (2035 bp).

FIG. 4 is a schematic illustration of the Gypsophila transformation vector pHAPAP.

FIGS. 5A-C are graphs showing the DNA content of cells taken from transgenic plantlets RP-1. RP-4 and RP-10 (FIG. 5A: RP-1. FIG. 5B: RP-4, FIG. 5C: RP-10).

FIGS. 6A-E are graphs showing the DNA content of cells taken from transgenic hybrid plantlets. (FIG. 6A: T.G-59. FIG. 6B: T.G-505, FIG. 6C: T.G-365, FIG. 6D: T.G-450. FIG. 6E: T.G-272).

FIG. 7 is a graphic presentation of Anthocyanins analysis of G. paniculata varieties (transgenic and non-transgenic).

FIG. 8 is a photomicrograph showing results of PCR analysis of transgenic Gypsophila. Controls include a molecular weight marker (L), negative control (non-transgenic Gypsophila extract; −CON) and positive control (+CON). Extracts from five different transgenic events are indicated in lanes TG59, TG272, TG505, TG365 and TG450.

FIG. 9 is a photomicrograph showing results of Southern analysis. Lanes shown are non-transgenic controls (MS and sample 7) and 8 transgenic gypsophila lines (lines (TG) 1, 4, 10, 59, 272, 365, 450 and 505). A positive control of pHAPAP plasmid DNA is included (+CON), along with a molecular weight ladder in the extreme right hand lane.

### DESCRIPTION OF SPECIFIC EMBODIMENTS OF THE INVENTION

The present invention, in some embodiments thereof, relates to Gypsophila paniculata plants.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

Gypsophila paniculata, also known as Baby's Breath, has long been valued as a filler plant in perennial border gardens and also as a long-lasting cut flower. Baby's Breath also makes an excellent dried flower. Due to its ornamental value, attempts have been made to generate varieties of Gypsophila paniculata incorporating novel and improved traits to the flower industry.

Thus, whilst reducing the present invention to practice, the present inventors were able, for the first time, to generate Gypsophila paniculata comprising a flower producing a stable, non-thermally induced, red, pink, purple or green pigmentation or a combination of same.

The present inventors have employed a novel method of transformation, which relies on the polyploidization of the M.S. variety and subjected the polyploid plant to genetic transformation for successfully expressing a heterologous PAP-1 polypeptide.

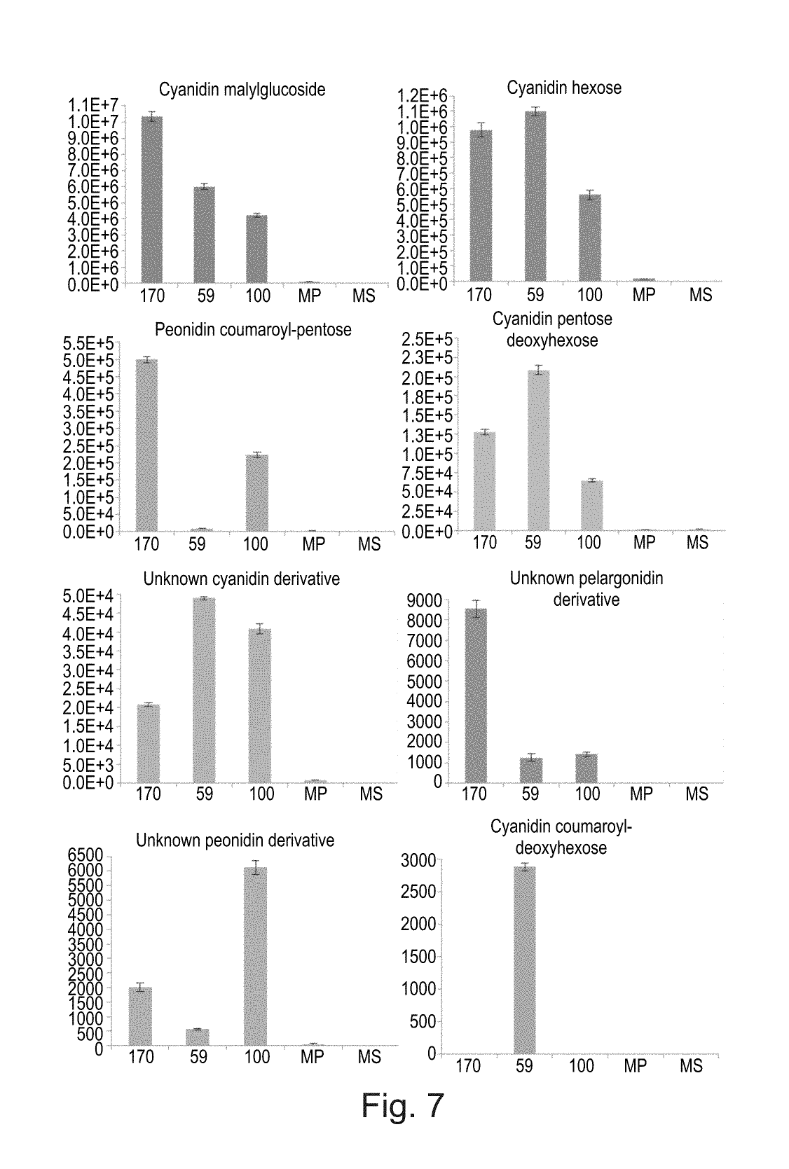

The resultant transformants exhibited a unique pigment profile as compared to the non-transformed plant of the same background. Thus, flower petals of the transformed plants exhibit non-thermally induced red, pink, purple or green pigmentation. These plants were used for the generation of hybrid lines. Hybrid production even broadened the pigmentation range of the flowers as shown in FIG. 7 and Table 4.

Thus, according to an aspect of the invention, there is provided a Gypsophila paniculata plant comprising a flower producing a non-thermally induced red, pink, purple, green pigmentation or any combination of same.

As used herein the term “plant” refers to a whole plant or parts thereof.

The phrase “plant part” refers to isolated plant cells or isolated plant parts (tissues) such as from which plants can be (re)generated, including plant protoplasts, plant cali, plant clumps, and plant cells that are intact in plants, or part of plants, such as seeds, leaves, stems, pollens, roots, root tips, anthers, ovules, petals, flowers, seedlings, embryos and bolls.

According to a specific embodiment the plant part is a flower.

According to a specific embodiment, the flower is a cut-flower.

According to a specific embodiment the plant part is a pollen.

According to a specific embodiment the plant part is an ovule.

According to a specific embodiment the plant part is a seed.

Also provided is a plant cutting (e.g., rooted or unrooted).

The plant may be any of the Gypsophila genus (as described below).

According to a specific embodiment, the plant is a Gypsophila paniculata plant or a hybrid having a pedigree which comprises Gypsophila paniculata.

As used herein “Gypsophila paniculata” also referred to as “Baby's Breath”, a cultivated plant of the Gypshophila genus.

Kingdom Plantae Plants

Subkingdom Tracheobionta Vascular plants

Superdivision Spermatoph—Seed plants

Division Magnoliophyta Flowering plants

Class Magnoliopsida Dicotyledons

Subclass Caryophyllidae

Order Caryophyllales

Family Caryophyllaceae Pink family

Genus Gypsophila L. baby's-breath:

The plant may have any of a desired habit, height and flower morphology.

Hence the plant height may be 20 cm to 1 meter. The growth habit may be compact, erect upright narrow or dense.

The flower type may be single, semi-double (SD) or double, as further described hereinbelow.

Semi-Double: a flower with more than one row of petals, and a clearly defined center which is visible.

Double: a flower with a few rows of petals, and a center which is not visible.

Double Multi-flowers: a flower with a few rows of petals, a center which is non-visible and developing young buds which are seen at the flower center.

Flower size: Small—smaller than 5 mm. Medium—between 6 to 9 mm. Large—between 9 to 30 mm.

According to a specific embodiment, the flower is a non-dyed flower.

Any known cultivar of Gypsophila paniculata or those that are constantly being developed are contemplated herein, including but not limited to. Arbel™, Bodgeri™, Bristol Fairy™, Compacta™, Compacta Plenar™, Dangypmini™, Dangysha™, Dansferoy™, Dantziger™, Snowflake™, Early Snowball™, Ehrlei™, Fairy Perfect™, Happy Festival™, Festival Pink™, Festival White™, Flamingo™, Floreplena™, Gilboa™, Golan™, Lucky Starts™, Million Stars™ (Dangypmini), Nana™, New Hope™, New Love™, Pacifica™, Perfecta™, Perfecta 53®, Perfecta Royal™, Pink Fairy™, Pink Star™, Plena™, Rahan 11™, Rahan 14™, Red Sea™, Romano 4™, Snowball™, Snowflake™, Snow White™, Tavor™, Viette's Dwarf™, Virgor™ and Yukinko™.

According to a specific embodiment, the plant does not have a pedigree (i.e., genetic background) of Arbel™, Pestival™ or Flamingo™.

According to a specific embodiment the plant has a pedigree which includes the genetic background of Million Stars™ or Million Stars™ (M.S.).

As mentioned the flowers of the plants of the present invention have red, pink, purple or green color.

According to a specific embodiment. “red” refers to RHS (2007): 59A, 60A, 30A, 33A, 34A, N34A-C, 35A, 37A-B, 39A-B, 40A-41C, 42A-43C, 44A-47B, 50A, 53A-C, 178A-179C, 180A-C, 181A-B, 182A-B.

According to a specific embodiment, “pink” refers to RHS (2007): 57A-D, 58B-D, 61C-62D, 63B-D, 64C-69D, 70C-D, 73A-D, 75C-D36A-D, 37C-38D, 39C-D, 41D, 43D, 47C-49D, 50B-52D, 53D-56D, 179D, 180D, 181C-D, 182C-D, 184C-D, 185C-D, White group: N155B-D.

According to a specific embodiment, the pink color is darker than that of 75B.

According to a specific embodiment. “purple” refers to RHS (2007): 58A, 59B-D, 60B-61B, 63A, 64A-B, 70A-B, 71A-72D, 74A-75B, 76A-88D, 183A-184B, 185A-B, 186A-N187D, White group: N155A.

According to a specific embodiment. “Green” refers to RHS (2007): Green-white group: 157A-D, Greyed-yellow group: 160C-D, Greyed-green group: 188A-N189B, 190A-196D. Blue-green group: 123D. 124C-D, Green group: 125A-143D, Yellow-green group: 144A-151D, 154A-D, Yellow group: 1A-C.

According to a specific embodiment, the flowers of the plants of the present invention have pink, red or purple color.

According to a specific embodiment, the flowers of the plants of the present invention have red or purple color.

According to a specific embodiment, the pigmentation is homogeneous in the petal, that is no specific pattern (e.g., stripes, dots spiral) is visible by human eye and or magnification lanes.

Alternatively, the color pattern may be non-homogenic, splash and/or comprise a distinct center and may include more than one color (e.g., 2, 3 or 4 colors).

It will be appreciated that according to some embodiments of the invention hybrids of the plants may have a color or color pattern which is not necessarily that of its ancestor. For example, where the ancestor comprises a pink, red, green or purple color its hybrid may have an orange or yellow color.

According to a specific embodiment, the pigmentation is non-temperature dependent, also referred to herein as “non thermally-induced”, that is, the above mentioned values of pigments are present in a basal level even at temperatures above 26° C. when in the field or as cut flowers.

According to a specific embodiment the plant has flower petals that contain cyanidin as the major anthocyanin (e.g., above 50%).

According to a specific embodiment the plant has flower petals that further comprise peonidin, and pelargonidin derivates.

According to a specific embodiment, the cyanidin comprises cyanidin malylglucoside and cyanidin hexose.

According to a specific embodiment the cyanidin malylglucoside and said cyanidin hexose are about 80-90% and 10-20% respectively, of total anthocyanin content of said flower petals, as assayed by UPLC-QTOF-MS.

The petals of the flowers of some embodiments of the invention are characterized by containing at least one of:

(i) at least about 10, 20, 50, 100 or 150 fold increase in cyanidin malylglucoside than that found in My Pink™ at the same developmental stage and assay conditions;

(ii) at least about 1,000, 2,000, 3000, 5,000, 8,000 or 10,000 fold increase in cyanidin malylglucoside than that found in Million Stars™ at the same developmental stage and assay conditions;

(iii) at least about 10, 20, 50, 100 or 150 fold increase in cyanidin hexose than that found in My Pink™ at the same developmental stage and assay conditions;

(iv) at least about 100, 200, 300, 500 or 1000 fold increase in peonidin coumaroyl pentose than that found in My Pink™ at the same developmental stage and assay conditions;

(v) at least about 50, 100, 200 or 500 fold increase in cyanidin pentose deoxyhexose than that found in My Pink™ at the same developmental stage and assay conditions.

According to a specific embodiment, the petals of the flowers of some embodiments of the invention are characterized by pigment profile reflected in (1)+(ii)+(iii)+(iv) and (v).

According to a specific embodiment, green flowers are characterized by increased levels of chlorophyll A (e.g., at least 1.2, 2, 3 or 4 folds) than that found in Million Stars™ at the same developmental stage and assay conditions; Alternatively or additionally, the green flowers are characterized by increased levels of chlorophyll B (e.g., at least 1.1, 2, 3 or 4 folds) than that found in Million Stars™ at the same developmental stage and assay conditions.

Indeed, as shown in FIG. 7, the anthocyanins identified in gypsophila petals generated according to the present teaches were different derivatives of cyanidin and peonidin, and unknown pelargonidin derivates. The major anthocyanin was cyanidin, with cyanidin malylglucoside and cyanidin hexose which comprised about 81-86% and 10-14% respectively, of the total anthocyanins content in the petals of the transgenic varieties tested. Minor amounts of peonidin coumaroyl-pentose and Cyanidine pentose deoxyhexose and un-known cyanidin derivatives were also identified, which comprised about 0.1-4% of the total anthocyanins content in the petals of the transgenic.

The major anthocyanins that were identified in My Pink™ M.P, the non transgenic light pink gypsophila, were also cyanidin malylglucoside and cyanidin hexose, which comprised about 82% and 14% respectively, of the total anthocyanins content in the petals. The major anthocyanins that were identified in M.S. the non transgenic white gypsophila, were cyanidin malylglucoside and cyanidine pentose deoxyhexose, which comprised about 44% and 55% respectively, of the total anthocyanins content in the petals.

Pigment analysis showed a range of anthocyanins amounts in the new varieties, ratios of cyanidin malylglucoside in variety 170 were 1.7 and 2.4 times higher compare to varieties 59 and 100, respectively. Ratios of cyanidin hexose in variety 170 were 0.9 and 1.7 times higher compare to varieties 59 and 100, respectively. Ratios of Peonidin coumaroyl-pentose in variety 170 were 54.7 and 2.2 times higher compare to varieties 59 and 100, respectively.

Methods of generating the plants of some embodiments of the invention include genetic modifications such as the introduction of a transgene encoding PAP1, upregulation of a silenced gene such as by the use of chimeric nucleases (CRISPR-Cas9, TALEN, Zinc-Finger nucleases, meganucleases etc. as described for instance in Gaj et al. Trends in Biotechnol. 2013 31(7):397-405; and also in WO2009/130695 each of which is herein incorporated by reference in its entirety) as well as classical breeding or the combination of same.

Though concentrating on genetic transformation/infection, the present teachings are not aiming to be limited to transgenic procedures.

Thus, according to an aspect of the invention there is provided a method of producing a transgenic Gypsophila paniculata comprising introducing into a Gypsophila paniculata plant a nucleic acid sequence encoding PAP1 operably linked to a cis-acting regulatory element active in a plant cell, thereby producing a transgenic Gypsophila paniculata.

As used herein the term “transgenic” refers to a plant which expresses a transgene, a nucleic acid sequence encoding an expression product (in this case PAP1 e.g., as set forth in SEQ ID NO: 2) which is not endogenous to the plant or plant cell.

According to a specific embodiment, the Gypsophila paniculata plant is subjected to a polyploidization protocol.

Accordingly, according to some embodiments of the invention, the polyploid Gypsophila paniculata has a higher chromosome number than the wild type Gypsophila paniculata (e.g., at least one chromosome set or portions thereof) such as for example two folds greater amount of genetic material (i.e., chromosomes) as compared to the wild type plant (e.g., M.S) and as described in Table 1 of the Examples section which follows. Induction of polyploidy is typically performed by subjecting a plant tissue to a G2/M cycle inhibitor.

Typically, the G2/M cycle inhibitor comprises a microtubule polymerization inhibitor.

Examples of microtubule cycle inhibitors include, but are not limited to oryzalin, colchicine, colcemid, trifluralin, benzimidazole carbamates (e.g. nocodazole, oncodazole, mebendazole, R 17934, MBC), o-isopropyl N-phenyl carbamate, chloroisopropyl N-phenyl carbamate, amiprophos-methyl, taxol, vinblastine, griseofulvin, caffeine, bis-ANS, maytansine, vinbalstine, vinblastine sulphate and podophyllotoxin.

Induction of polyploidy can be performed on the wild-type plant, as described in Example 1 or the cultivated plant that exhibits new flower color, the latter is mainly done to improve horticultural traits.

According to a specific embodiment, the Gypsophila paniculata plant has a M.S polyploidy genetic background. In other words the source material for introducing the PAP1 transgene is the M.S polyploid Gypsophila paniculata plant.

As used herein “PAP1” refers to the gene product, the transcription factor MYB75 having the gene symbol PAP1 and able to increase the anthocyanin content in the Gypsophila paniculata plant, e.g., in flower petals, stem, stem leaves, pollen, anthers, pistils, ovaries, sepals.

According to a specific embodiment, the anthocyanin accumulation is at least in (i.e., not limited to) the flower.

According to a specific embodiment, the anthocyanin accumulation is not in the flower. In such a case, the flowers will be white.

The gene is also known as ATMYB75; F25P12.92; F25P12_92; MYB DOMAIN PROTEIN 75; MYB75; MYELOBLASTOSIS PROTEIN 75; PAP1; phosphatidic acid phosphatase 1; production of anthocyanin pigment 1; SIAA1; SUC-INDUCED ANTHOCYANIN ACCUMULATION 1 and encodes a putative MYB domain containing transcription factor involved in anthocyanin metabolism and radical scavenging.

According to a specific embodiment the PAP1 is exogenous to the Gypsophila and is integrated in the genome of the transgenic plant, also referred to herein as stable transformation.

As used herein the term “exogenous” refers to a nucleic acid sequence which is not present in wild-type Gypsophila of the same genetic background.

According to a specific embodiment, the exogenous PAP1 is present in the genome of the Gypsophila in multiple copies (also referred to as “integration events”).

According to a specific embodiment, the PAP1 is present in the Gypsophila genome in at least two copies.

According to a specific embodiment, the PAP1 is present in the Gypsophila genome in at least three copies.

According to a specific embodiment, the PAP1 is present in the Gypsophila genome in at least four copies.

According to a specific embodiment, the PAP1 is present in the Gypsophila genome in at least five copies.

According to a specific embodiment the PAP1 is of the Arabidopsis thaliana GenBank accession no. AF325123.

According to a specific embodiment, PAP1 is as set forth in SEQ ID NO: 2 or a homolog of said SEQ ID NO: 2 encoded by SEQ ID NO: 1 or homologs thereof.

The understanding that PAP1 activation can change the color of Gypsophila paniculata and induce anthocyanin accumulation in the plant, suggests that such plants can also be obtained by classical breeding using the Gypsophila PAP1 homolog as a selection marker (other selection marker may be the pigmentation).

Thus, contemplated are polynucleotide sequences and polypeptide sequences which are homologous to SEQ ID NO: 1 or 2 as long as the function of the expression product (increase the anthocyanin content in petals of the Gypsophila paniculata plant) and optionally its stability are maintained.

Thus, according to a specific embodiment, the amino acid sequence of the PAP1 polypeptide is at least about 60%, at least about 70%, at least about 80%, at least about 85%; at least about 90%, at least about 95%, at least about 98%, at least about 99% or 100% identical or homologous to SEQ ID NO: 2 as long as the function of the expression product and optionally its stability are maintained.

Alternatively or additionally, the nucleic acid sequence of the PAP1 polynucleotide is at least about 60%, at least about 70%, at least about 80%, at least about 85%; at least about 90%, at least about 95%, at least about 98%, at least about 99% or 100% identical to SEQ ID NO: 1 as long as the function of the expression product and optionally its stability are maintained.

As used herein, the terms “polynucleotide” and “nucleic acid sequence”, which are interchangeably used, refer to a single or double stranded nucleic acid sequence which is isolated and provided in the form of an RNA sequence, a complementary polynucleotide sequence (cDNA), a genomic polynucleotide sequence and/or a composite polynucleotide sequences (e.g., a combination of the above).

As used herein the phrase “complementary polynucleotide sequence” refers to a sequence, which results from reverse transcription of messenger RNA using a reverse transcriptase or any other RNA dependent DNA polymerase. Such a sequence can be subsequently amplified in vivo or in vitro using a DNA dependent DNA polymerase.

As used herein the phrase “genomic polynucleotide sequence” refers to a sequence derived (isolated) from a chromosome and thus it represents a contiguous portion of a chromosome.

As used herein the phrase “composite polynucleotide sequence” refers to a sequence, which is at least partially complementary and at least partially genomic. A composite sequence can include some exonal sequences required to encode the polypeptide of the present invention, as well as some intronic sequences interposing therebetween. The intronic sequences can be of any source, including of other genes, and typically will include conserved splicing signal sequences. Such intronic sequences may further include cis acting expression regulatory elements, as described in further detail below.

Nucleic acid sequences of the polypeptides of some embodiments of the invention may be optimized for plant expression. Examples of such sequence modifications include, but are not limited to, an altered G/C content to more closely approach that typically found in the plant species of interest, and the removal of codons atypically found in the plant species commonly referred to as codon optimization.

The phrase “codon optimization” refers to the selection of appropriate DNA nucleotides for use within a structural gene or fragment thereof that approaches codon usage within the plant of interest. Therefore, an optimized gene or nucleic acid sequence refers to a gene in which the nucleotide sequence of a native or naturally occurring gene has been modified in order to utilize statistically-preferred or statistically-favored codons within the plant. The nucleotide sequence typically is examined at the DNA level and the coding region optimized for expression in the plant species determined using any suitable procedure, for example as described in Sardana et al. (1996, Plant Cell Reports 15:677-681). In this method, the standard deviation of codon usage, a measure of codon usage bias, may be calculated by first finding the squared proportional deviation of usage of each codon of the native gene relative to that of highly expressed plant genes, followed by a calculation of the average squared deviation. The formula used is: 1 SDCU=n=1 N [(Xn−Yn)/Yn]2/N, where Xn refers to the frequency of usage of codon n in highly expressed plant genes, where Yn to the frequency of usage of codon n in the gene of interest and N refers to the total number of codons in the gene of interest. A table of codon usage from highly expressed genes of dicotyledonous plants is compiled using the data of Murray et al. (1989, Nuc Acids Res. 17:477-498).

Thus, embodiments of the invention encompass nucleic acid sequences described hereinabove; fragments thereof, sequences hybridizable therewith, sequences homologous thereto, sequences orthologous thereto, sequences encoding similar polypeptides with different codon usage, altered sequences characterized by mutations, such as deletion, insertion or substitution of one or more nucleotides, either naturally occurring or man induced, either randomly or in a targeted fashion.

Constructs useful in the methods according to the present invention may be constructed using recombinant DNA technology well known to persons skilled in the art. The gene constructs may be inserted into vectors, which may be commercially available, suitable for transforming into e.g. plants and suitable for expression of the gene of interest in the transformed cells. The genetic construct can be an expression vector whereby, as mentioned, the heterologous nucleic acid sequence is operably linked to a cis-acting regulatory element allowing expression in the cells, such as in plant cells.

As used herein, the phrase “cis acting regulatory element” refers to a polynucleotide sequence, preferably a promoter, which binds a trans acting regulator and regulates the transcription of a coding sequence located downstream thereto.

According to specific embodiments the cis-acting regulatory element comprises a promoter sequence.

As used herein, the phrase “operably linked” refers to a functional positioning of the cis-regulatory element (e.g., promoter) so as to allow regulating expression of the selected nucleic acid sequence. For example, a promoter sequence may be located upstream of the selected nucleic acid sequence in terms of the direction of transcription and translation (e.g., of PAP1).

According to an embodiment, the promoter in the nucleic acid construct of the present invention is a plant promoter which serves for directing expression of the heterologous nucleic acid molecule within plant cells.

As used herein the phrase “plant promoter” refers to a promoter sequence, including any additional regulatory elements added thereto or contained therein, is at least capable of inducing, conferring, activating or enhancing expression in a plant cell, tissue or organ, preferably a woody plant cell, tissue, or organ. Such a promoter can be derived from a plant, bacterial, viral, fungal or animal origin. Such a promoter can be constitutive, i.e., capable of directing high level of gene expression in a plurality of plant tissues, tissue specific, i.e., capable of directing gene expression in a particular plant tissue (e.g., flower petals) or tissues, inducible, i.e., capable of directing gene expression under a stimulus, or chimeric. i.e., formed of portions of at least two different promoters.

According to specific embodiments the promoter is a constitutive promoter.

Examples of constitutive plant promoters include, without being limited to. CaMV35S and CaMV19S promoters, Figwort mosaic virus subgenomic transcript (sgFiMV) promoter, Strawberry vein banding virus (SVBV) promoter, FMV34S promoter, sugarcane bacilliform badnavirus promoter, CsVMV promoter, Arabidopsis ACT2/ACT8 actin promoter. Arabidopsis ubiquitin UBQ1 promoter, barley leaf thionin BTH6 promoter, and rice actin promoter.

According to a specific embodiment the constitutive promoter is Cauliflower mosaic virus (CaMV) 35S promoter.

Other exemplary promoters useful for the methods of some embodiments of the invention are presented in Tables I, II and III.

TABLE I
Exemplary constitutive promoters for use in the
performance of some embodiments of the invention

| | Expression | |
|---|---|---|
| Gene Source | Pattern | Reference |
| Actin | constitutive | McElroy et al, Plant Cell, |
| | | 2: 163-171, 1990 |
| CAMV 35S | constitutive | Odell et al, Nature, 313: |
| | | 810-812, 1985 |
| CaMV 19S | constitutive | Nilsson et al., Physiol. |
| | | Plant 100: 456-462, 1997 |
| GOS2 | constitutive | de Pater et al, Plant J |
| | | Nov; 2(6): 837-44, 1992 |
| ubiquitin | constitutive | Christensen et al, Plant |
| | | Mol. Biol. 18: 675-689, |
| | | 1992 |
| Rice cyclophilin | constitutive | Bucholz et al, Plant Mol |
| | | Biol. 25(5): 837-43, 1994 |
| Maize H3 histone | constitutive | Lepetit et al, Mol. Gen. |
| | | Genet. 231: 276-285, |
| | | 1992 |
| Actin 2 | constitutive | An et al, Plant J. |
| | | 10(1); 107-121, 1996 |

TABLE II
Exemplary seed-preferred promoters for use in the
performance of some embodiments of the invention

| | Expression | |
|---|---|---|
| Gene Source | Pattern | Reference |
| Seed specific genes | seed | Simon, et al., Plant Mol. |
| | | Biol, 5. 191, 1985; |
| | | Scofield, |
| | | et al., J. Biol. Chem. |
| | | 262: 12202, 1987.; |
| | | Baszczynski, et al., Plant |
| | | Mol. Biol. 14: 633, 1990. |
| Brazil Nut albumin | seed | Pearson' et al., Plant Mol. |
| | | Biol. 18: 235-245, 1992. |
| legumin | seed | Ellis, et al. Plant Mol. |
| | | Biol. 10: 203-214, 1988 |
| Glutelin (rice) | seed | Takaiwa, et al., Mol. Gen, |
| | | Genet. 208: 15-22, 1986; |
| | | Takaiwa, et al., FEBS |
| | | Letts. 221: 43-47, 1987 |
| Zein | seed | Matzke et al. Plant Mol |
| | | Biol, 143). 323-32 1990 |
| napA | seed | Stalberg, et al., Planta |
| | | 199: 515-519, 1996 |
| wheat, LMW and HMW, | endosperm | Mol Gen Genet 216: 81- |
| glutenin-1 | | 90, 1989; NAR 17: 461-2, |
| Wheat SPA | seed | Albanietal, Plant Cell, 9: |
| | | 171-184, 1997 |
| wheat a, b and g gliadins | endosperm | EMBO3: 1409-15, 1984 |
| Barley ltrl promoter | endosperm | |
| barley B1, C, D hordein | endosperm | Theor Appl Gen 98: 1253- |
| | | 62, 1999; Plant J 4: 343- |
| | | 55, 1993; Mol Gen Genet |
| | | 250: 750- 60, 1996 |
| Barley DOF | endosperm | Mena et al., The Plant |
| | | Journal, 116(1): 53-62, |
| | | 1998 |
| Biz2 | endosperm | EP99106056.7 |
| Synthetic promoter | endosperm | Vicente-Carbajosa et al., |
| | | Plant J. 13: 629-640. 1998 |
| rice prolamin NRP33 | endosperm | Wu et al., Plant Cell |
| | | Physiology 39(8) 885- |
| | | 889, 1998 |
| rice -globulin Glb-1 | endosperm | Wu et al., Plant Cell |
| | | Physiology 398) 885-889, |
| | | 1998 |
| rice OSH1 | embryo | Sato et al., Proc. Nati. |
| | | Acad. Sci. USA, 93: |
| | | 8117-8122 |
| rice alpha-globulin | endosperm | Nakase et al. Plant Mol. |
| REB/OHP-1 | | Biol. 33: 513-S22, 1997 |
| rice ADP-glucose PP | endosperm | Trans Res 6: 157-68, 1997 |
| maize ESR gene family | endosperm | Plant J 12: 235-46, 1997 |
| sorgum gamma- kafirin | endosperm | PMB 32: 1029-35, 1996 |
| KNOX | embryo | Postma-Haarsma et al., |
| | | Plant Mol. Biol. 39: 257- |
| | | 71, 1999 |
| rice oleosin | Embryo and | Wu et al., J. Biochem., |
| | aleuton | 123: 386, 1998 |
| sunflower oleosin | Seed (embryo | Cummins, et al., Plant |
| | and dry seed) | Mol. Biol. 19: 873-876, |
| | | 1992 |

TABLE III
Exemplary flower-specific promoters for
use in the performance of the invention

| | Expression | |
|---|---|---|
| Gene Source | Pattern | Reference |
| AtPRP4 | flowers | www.salus. |
| | | medium.edu/m |
| | | mg/tierney/html |
| chalene synthase | flowers | Van der Meer, et al., Plant |
| (chsA) | | Mol. Biol. 15, 95- |
| | | 109, 1990. |
| LAT52 | anther | Twell et al., Mol. Gen |
| | | Genet. 217: 240-245 |
| | | (1989) |
| apetala- 3 | flowers | |

Plant cells may be transformed stably or transiently with the nucleic acid constructs of some embodiments of the invention. In stable transformation, the nucleic acid molecule of some embodiments of the invention is integrated into the plant genome and as such it represents a stable and inherited trait. In transient transformation, the nucleic acid molecule is expressed by the cell transformed but it is not integrated into the genome and as such it represents a transient trait.

There are various methods of introducing foreign genes into both monocotyledonous and dicotyledonous plants (Potrykus, I., Annu. Rev. Plant. Physiol., Plant. Mol. Biol. (1991) 42:205-225: Shimamoto et al., Nature (1989) 338:274-276).

According to a specific embodiment, the transgene is introduced into the plant by seed transformation (as described in the Examples section which follows), yet introduction to cuttings may also be possible, as described in Moyal Ben Zvi, M., Zuker. A., Ovadis, M., Shklarman, E., Ben-Meir, H., Zenvirt, S. and Vainstein, A. (2008) Agrobacterium-mediated transformation of gypsophila (Gypsophila paniculata L.) Mol. Breeding 22:543-553.

The principle methods of causing stable integration of exogenous DNA into plant genomic DNA include two main approaches:

(i) Agrobacterium-mediated gene transfer: Klee et al. (1987) Annu. Rev. Plant Physiol. 38:467-486; Klee and Rogers in Cell Culture and Somatic Cell Genetics of Plants. Vol. 6, Molecular Biology of Plant Nuclear Genes, eds. Schell, J., and Vasil, L. K., Academic Publishers, San Diego, Calif. (1989) p. 2-25; Gatenby, in Plant Biotechnology, eds. Kung. S. and Arntzen, C. J., Butterworth Publishers, Boston, Mass. (1989) p. 93-112.

(ii) direct DNA uptake: Paszkowski et al., in Cell Culture and Somatic Cell Genetics of Plants, Vol. 6. Molecular Biology of Plant Nuclear Genes eds. Schell, J., and Vasil. L. K., Academic Publishers. San Diego, Calif. (1989) p. 52-68; including methods for direct uptake of DNA into protoplasts. Toriyama, K. et al. (1988) Bio/Technology 6:1072-1074. DNA uptake induced by brief electric shock of plant cells: Zhang et al. Plant Cell Rep. (1988) 7:379-384. Fromm et al. Nature (1986) 319:791-793. DNA injection into plant cells or tissues by particle bombardment, Klein et al. Bio/Technology (1988) 6:559-563; McCabe et al. Bio/Technology (1988) 6:923-926; Sanford, Physiol. Plant. (1990) 79:206-209; by the use of micropipette systems: Neuhaus et al., Theor. Appl. Genet. (1987) 75:30-36; Neuhaus and Spangenberg, Physiol. Plant. (1990) 79:213-217; glass fibers or silicon carbide whisker transformation of cell cultures, embryos or callus tissue, U.S. Pat. No. 5,464,765 or by the direct incubation of DNA with germinating pollen. DeWet et al. in Experimental Manipulation of Ovule Tissue, eds. Chapman, G. P. and Mantell. S. H. and Daniels. W. Longman, London. (1985) p. 197-209; and Ohta, Proc. Natl. Acad. Sci. USA (1986) 83:715-719.

The Agrobacterium system includes the use of plasmid vectors that contain defined DNA segments that integrate into the plant genomic DNA. Methods of inoculation of the plant tissue vary depending upon the plant species and the Agrobacterium delivery system. A widely used approach is the leaf disc procedure which can be performed with any tissue explant that provides a good source for initiation of whole plant differentiation. Horsch et al. in Plant Molecular Biology Manual A5, Kluwer Academic Publishers, Dordrecht (1988) p. 1-9. A supplementary approach employs the Agrobacterium delivery system in combination with vacuum infiltration. The Agrobacterium system is especially viable in the creation of transgenic dicotyledonous plants.

There are various methods of direct DNA transfer into plant cells. In electroporation, the protoplasts are briefly exposed to a strong electric field. In microinjection, the DNA is mechanically injected directly into the cells using very small micropipettes. In microparticle bombardment, the DNA is adsorbed on microprojectiles such as magnesium sulfate crystals or tungsten particles, and the microprojectiles are physically accelerated into cells or plant tissues.

Following stable transformation plant propagation is exercised. The most common method of plant propagation is by seed. Regeneration by seed propagation, however, has the deficiency that due to heterozygosity there is a lack of uniformity in the crop, since seeds are produced by plants according to the genetic variances governed by Mendelian rules. Basically, each seed is genetically different and each will grow with its own specific traits. Therefore, it is preferred that the transformed plant be produced such that the regenerated plant has the identical traits and characteristics of the parent transgenic plant. Therefore, it is preferred that the transformed plant be regenerated by micropropagation which provides a rapid, consistent reproduction of the transformed plants.

Micropropagation is a process of growing new generation plants from a single piece of tissue that has been excised from a selected parent plant or cultivar. This process permits the mass reproduction of plants having the preferred tissue expressing the fusion protein. The new generation plants which are produced are genetically identical to, and have all of the characteristics of, the original plant. Micropropagation allows mass production of quality plant material in a short period of time and offers a rapid multiplication of selected cultivars in the preservation of the characteristics of the original transgenic or transformed plant. The advantages of cloning plants are the speed of plant multiplication and the quality and uniformity of plants produced.

Micropropagation is a multi-stage procedure that requires alteration of culture medium or growth conditions between stages. Thus, the micropropagation process involves four basic stages: Stage one, initial tissue culturing; stage two, tissue culture multiplication; stage three, differentiation and plant formation; and stage four, greenhouse culturing and hardening. During stage one, initial tissue culturing, the tissue culture is established and certified contaminant-free. During stage two, the initial tissue culture is multiplied until a sufficient number of tissue samples are produced to meet production goals. During stage three, the tissue samples grown in stage two are divided and grown into individual plantlets. At stage four, the transformed plantlets are transferred to a greenhouse for hardening where the plants' tolerance to light is gradually increased so that it can be grown in the natural environment.

Although stable transformation is presently preferred, transient transformation of leaf cells, meristematic cells or the whole plant is also envisaged by some embodiments of the invention.

Transient transformation can be effected by any of the direct DNA transfer methods described above or by viral infection using modified plant viruses.

Viruses that have been shown to be useful for the transformation of plant hosts include CaMV. TMV and BV. Transformation of plants using plant viruses is described in U.S. Pat. No. 4,855,237 (BGV), EP-A 67,553 (TMV), Japanese Published Application No. 63-14693 (TMV), EPA 194,809 (BV). EPA 278.667 (BV); and Gluzman, Y. et al., Communications in Molecular Biology: Viral Vectors, Cold Spring Harbor Laboratory, New York, pp. 172-189 (1988). Pseudovirus particles for use in expressing foreign DNA in many hosts, including plants, is described in WO 87/06261.

Construction of plant RNA viruses for the introduction and expression of non-viral exogenous nucleic acid sequences in plants is demonstrated by the above references as well as by Dawson, W. O. et al., Virology (1989) 172:285-292; Takamatsu et al. EMBO J. (1987) 6:307-311; French et al. Science (1986) 231:1294-1297; and Takamatsu et al. FEBS Letters (1990) 269:73-76.

When the virus is a DNA virus, suitable modifications can be made to the virus itself. Alternatively, the virus can first be cloned into a bacterial plasmid for ease of constructing the desired viral vector with the foreign DNA. The virus can then be excised from the plasmid. If the virus is a DNA virus, a bacterial origin of replication can be attached to the viral DNA, which is then replicated by the bacteria. Transcription and translation of this DNA will produce the coat protein which will encapsidate the viral DNA. If the virus is an RNA virus, the virus is generally cloned as a cDNA and inserted into a plasmid. The plasmid is then used to make all of the constructions. The RNA virus is then produced by transcribing the viral sequence of the plasmid and translation of the viral genes to produce the coat protein(s) which encapsidate the viral RNA.

Construction of plant RNA viruses for the introduction and expression in plants of non-viral exogenous nucleic acid sequences such as those included in the construct of some embodiments of the invention is demonstrated by the above references as well as in U.S. Pat. No. 5,316,931.

In one embodiment, a plant viral nucleic acid is provided in which the native coat protein coding sequence has been deleted from a viral nucleic acid, a non-native plant viral coat protein coding sequence and a non-native promoter, preferably the subgenomic promoter of the non-native coat protein coding sequence, capable of expression in the plant host, packaging of the recombinant plant viral nucleic acid, and ensuring a systemic infection of the host by the recombinant plant viral nucleic acid, has been inserted. Alternatively, the coat protein gene may be inactivated by insertion of the non-native nucleic acid sequence within it, such that a protein is produced. The recombinant plant viral nucleic acid may contain one or more additional non-native subgenomic promoters. Each non-native subgenomic promoter is capable of transcribing or expressing adjacent genes or nucleic acid sequences in the plant host and incapable of recombination with each other and with native subgenomic promoters. Non-native (foreign) nucleic acid sequences may be inserted adjacent the native plant viral subgenomic promoter or the native and a non-native plant viral subgenomic promoters if more than one nucleic acid sequence is included. The non-native nucleic acid sequences are transcribed or expressed in the host plant under control of the subgenomic promoter to produce the desired products.

In a second embodiment, a recombinant plant viral nucleic acid is provided as in the first embodiment except that the native coat protein coding sequence is placed adjacent one of the non-native coat protein subgenomic promoters instead of a non-native coat protein coding sequence.

In a third embodiment, a recombinant plant viral nucleic acid is provided in which the native coat protein gene is adjacent its subgenomic promoter and one or more non-native subgenomic promoters have been inserted into the viral nucleic acid. The inserted non-native subgenomic promoters are capable of transcribing or expressing adjacent genes in a plant host and are incapable of recombination with each other and with native subgenomic promoters. Non-native nucleic acid sequences may be inserted adjacent the non-native subgenomic plant viral promoters such that said sequences are transcribed or expressed in the host plant under control of the subgenomic promoters to produce the desired product.

In a fourth embodiment, a recombinant plant viral nucleic acid is provided as in the third embodiment except that the native coat protein coding sequence is replaced by a non-native coat protein coding sequence.

The viral vectors are encapsidated by the coat proteins encoded by the recombinant plant viral nucleic acid to produce a recombinant plant virus. The recombinant plant viral nucleic acid or recombinant plant virus is used to infect appropriate host plants. The recombinant plant viral nucleic acid is capable of replication in the host, systemic spread in the host, and transcription or expression of foreign gene(s) (isolated nucleic acid) in the host to produce the desired protein.

In addition to the above, the nucleic acid molecule of some embodiments of the invention can also be introduced into a chloroplast genome thereby enabling chloroplast expression.

A technique for introducing exogenous nucleic acid sequences to the genome of the chloroplasts is known. This technique involves the following procedures. First, plant cells are chemically treated so as to reduce the number of chloroplasts per cell to about one. Then, the exogenous nucleic acid is introduced via particle bombardment into the cells with the aim of introducing at least one exogenous nucleic acid molecule into the chloroplasts. The exogenous nucleic acid is selected such that it is integratable into the chloroplast's genome via homologous recombination which is readily effected by enzymes inherent to the chloroplast. To this end, the exogenous nucleic acid includes, in addition to a gene of interest, at least one nucleic acid stretch which is derived from the chloroplast's genome. In addition, the exogenous nucleic acid includes a selectable marker, which serves by sequential selection procedures to ascertain that all or substantially all of the copies of the chloroplast genomes following such selection will include the exogenous nucleic acid. Further details relating to this technique are found in U.S. Pat. Nos. 4,945,050; and 5,693,507 which are incorporated herein by reference. A polypeptide can thus be produced by the protein expression system of the chloroplast and become integrated into the chloroplast's inner membrane.

Regardless of the method of production, the (transgenic) plant (having flowers producing a stable non-thermally induced red, pink, purple or green pigmentation or a combination of same as described herein e.g., expressing exogenous PAP1) is selected according to the flower color as defined above.

Thus, there is provided a Gypsophila paniculata plant comprising an exogenous nucleic acid sequence encoding PAP1.

The plants of some embodiments of the invention may be polyploid, e.g., tetraploid as taught in Example 1 for instance.

The present invention also contemplates crossing the plants with other Gypsophila species (e.g., Gypsophila paniculata) to generate hybrid or inbred lines.

As used herein “Gypsophila” refers to a genus of flowering plants in the carnation family, Caryophyllaceae. There are about 150 species in the genus, some are provided hereinbelow.

Gypsophila acutifolia—sharpleaf baby's-breath
Gypsophila altissima
Gypsophila aretioides
Gypsophila arrostii—Arrost's baby's-breath
Gypsophila bicolor
Gypsophila capituliflora
Gypsophila cephalotes
Gypsophila cerastioides—chickweed baby's-breath
Gypsophila davurica
Gypsophila desertorum
Gypsophila elegans—showy baby's-breath
Gypsophila fastigiata—fastigiate gypsophila
Gypsophila glandulosa
Gypsophila glomerata
Gypsophila huashanensis
Gypsophila libanotica
Gypsophila licentiana
Gypsophila muralis—annual gypsophila, cushion baby's-breath, low baby's-breath
Gypsophila nana—dwarf gypsophila
Gypsophila oldhamiana—Manchurian baby's-breath, Oldham's baby's-breath
Gypsophila pacifica
Gypsophila paniculata—baby's-breath, common gypsophila, panicled baby's-breath
Gypsophila patrinii
Gypsophila perfoliata—perfoliate gypsophila
Gypsophila petraea
Gypsophila pilosa—Turkish baby's-breath
Gypsophila repens—alpine gypsophila, creeping baby's-breath
Gypsophila rokejeka
Gypsophila ruscifolia
Gypsophila scorzonerifolia—glandular baby's-breath, garden baby's-breath
Gypsophila sericea
Gypsophila silenoides
Gypsophila spinosa
Gypsophila stevenii—Steven's baby's-breath
Gypsophila struthium
Gypsophila tenuifolia
Gypsophila tschiliensis
Gypsophila uralensis
Gypsophila venusta
Gypsophila viscosa

Once established. Gypsophila paniculata plants and lines can be propagated by using tissue culturing techniques.

As used herein the phrase “tissue culture” refers to plant cells or plant parts from which Gypsophila paniculata plants can be generated, including plant protoplasts, plant cali, plant clumps, and plant cells that are intact in plants, or part of plants, such as seeds, leaves, stems, pollens, roots, root tips, anthers, ovules, petals, flowers and embryos.

Techniques of generating plant tissue culture and regenerating plants from tissue culture are well known in the art. For example, such techniques are set forth by Vasil (1984) [Cell Culture and Somatic Cell Genetics of Plants, Vol I, II. III Laboratory Procedures and Their Applications Academic Press, New York]; Green et al. (1987) [Plant Tissue and Cell Culture. Academic Press, New York]; Weissbach and Weissbach (1989) [Methods for Plant Molecular Biology. Academic Press]: Gelvin et al. (1990) [Plant Molecular Biology Manual. Kluwer Academic Publishers]: Evans et al. (1983) [Handbook of Plant Cell Culture, MacMillian Publishing Company, New York]; and Klee et al. (1987) [Ann. Rev. of Plant Phys. 38:467-486].

As mentions, the plant having the unique pigmentation may also comprise unique pigmentation in plant parts which are not limited to the flower petals e.g., leaves and stems anthers, pistils, ovaries, sepals.

Thus, according to a specific embodiment, the plant part is selected from the group consisting of a leaf, anther, stem, sepal and pistil and wherein the plant part exhibits a cyanidin level higher than that found in Gypsophyla paniculata var. Million Starts™ or My Pink™ being of the same developmental stage and growth conditions.

The plants of some embodiments of the invention may be further bred to comprise a horticultural favorable trait, for example, vase life, disease resistance, flower shape, plant habit, pot plant gypsophila and day-naturalize plants.

Thus, there is provided a method of developing a cultivated plant using plant breeding techniques, the method comprising using the plant or plant part as described herein (having flowers producing a non-thermally induced red, pink, purple or green pigmentation or a combination of same as described herein, e.g., expressing exogenous PAP1) as a source of breeding material for self-breeding and/or cross-breeding.

More specifically, there is provided a method of producing a Gypsophila paniculata plant, the method comprising:

(a) crossing the plant or plant part as described herein (having flowers producing a non-thermally induced red, pink, purple or green pigmentation or a combination of same as described herein e.g., expressing exogenous PAP1) with another Gypsophila plant e.g., Gypsophila paniculata plant;
(b) recovering seeds following said crossing;
(c) planting said seeds and growing said seed into plants; and
(d) selecting a hybrid plant.

According to a specific embodiment selecting is according to pigmentation.

Thus, the plant or plant part as described herein (having flowers producing a stable non-thermally induced red, pink, purple or green pigmentation or a combination of same as described herein e.g., expressing exogenous PAP1) may be subjected to recurrent selection, pedigree breeding and/or backcrossing to generate unique progeny or parental lines most suitable for final progeny production.

Additional screening techniques including restriction fragment length polymorphism selection or genetic marker selection (e.g., PAP1) can also be used to further facilitate progeny selection.

Thus, the present teachings also provide for hybrid plants, hybrid seeds, inbred plants, inbred seeds each of which may be polyploid or have a wild-type ploidy.

Employing the teachings of some embodiments of the invention, the present invention have generated a number of Gypsophila paniculata lines such as 170, 100 hand 59, as well as T.G-59, T.G-505, T.G-365, T.G-450. T.G-272.

According to a specific embodiment, the plant has essentially all the characteristics of T.G-505.

According to a specific embodiment, the plant has essentially all the characteristics of T.G-59.

Thus, also provided is a Gypsophila paniculata plant comprising a flower producing a non-thermally induced red, pink, purple or green pigmentation or a combination of same, wherein a sample of representative seeds of Gypsophila paniculata plant comprising a flower producing a non-thermally induced red, pink, purple or green pigmentation or a combination of same is deposited.

Seeds of the plants of the present invention may be seeded and therefore the present invention contemplates a sawn field. Vegetative portions of the plants of the invention can be planted. Thus the present invention also contemplates a planted field or a potted plant.

The plant cutting can be placed in a container (such as a growth cell, a plug) which contains the plant cutting and therefore the present invention also contemplates the holding vessel which comprises the cuttings. The plant cutting may be rooted or unrooted.

The cut flowers can be placed in a container (such as a vase, a bucket or pail or another holding apparatus) which contains the cut flowers and therefore the present invention also contemplates the holding vessel which comprises the cuttings.

The plants of the invention can also be rooted, grown or held in a container with other plant species (such as having at least one growth characteristic e.g., rooting time, growth rate) for the display of multispecies combinations (also referred to in the art as combos, Mixies™, Trixies™ and the like). Such configurations are taught for example in U.S. Pat. No. 8,136,294, which is hereby incorporated by reference in its entirety.

It is expected that during the life of a patent maturing from this application many relevant Gypsophila paniculata will be developed and the scope of the term Gypsophila paniculata is intended to include all such new technologies a priori.

As used herein the term “about” refers to +10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/for parts, but only if the additional ingredients, steps and/or parts do not materially alter the basic and novel characteristics of the claimed composition, method or structure.

As used herein, the singular form “a”. “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

When reference is made to particular sequence listings, such reference is to be understood to also encompass sequences that substantially correspond to its complementary sequence as including minor sequence variations, resulting from, e.g., sequencing errors, cloning errors, or other alterations resulting in base substitution, base deletion or base addition, provided that the frequency of such variations is less than 1 in 50 nucleotides, alternatively, less than 1 in 100 nucleotides, alternatively, less than 1 in 200 nucleotides, alternatively, less than 1 in 500 nucleotides, alternatively, less than 1 in 1000 nucleotides, alternatively, less than 1 in 5,000 nucleotides, alternatively, less than 1 in 10,000 nucleotides.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non limiting fashion.

Generally, the nomenclature used herein and the laboratory procedures utilized in the present invention include molecular, biochemical, microbiological and recombinant DNA techniques. Such techniques are thoroughly explained in the literature. See, for example, “Molecular Cloning: A laboratory Manual” Sambrook et al., (1989); “Current Protocols in Molecular Biology” Volumes 1-III Ausubel. R. M., ed. (1994); Ausubel et al., “Current Protocols in Molecular Biology”, John Wiley and Sons, Baltimore, Md. (1989); Perbal, “A Practical Guide to Molecular Cloning”, John Wiley & Sons, New York (1988); Watson et al., “Recombinant DNA”, Scientific American Books, New York; Birren et al. (eds) “Genome Analysis: A Laboratory Manual Series”. Vols. 1-4, Cold Spring Harbor Laboratory Press, New York (1998); methodologies as set forth in U.S. Pat. Nos. 4,666,828; 4,683,202; 4,801,531; 5,192,659 and 5,272,057; “Cell Biology: A Laboratory Handbook”. Volumes I-III Cellis, J. E., ed. (1994); “Current Protocols in Immunology” Volumes I-III Coligan J. E., ed. (1994); Stites et al. (eds), “Basic and Clinical Immunology” (8th Edition), Appleton & Lange, Norwalk, Conn. (1994); Mishell and Shiigi (eds), “Selected Methods in Cellular Immunology”, W. H. Freeman and Co., New York (1980); available immunoassays are extensively described in the patent and scientific literature, see, for example, U.S. Pat. Nos. 3,791,932; 3,839,153; 3,850,752; 3,850,578; 3,853,987; 3,867,517; 3,879,262; 3,901,654; 3,935,074; 3,984,533; 3,996,345; 4,034,074; 4,098,876; 4,879,219; 5,011,771 and 5,281,521; “Oligonucleotide Synthesis” Gait, M. J., ed. (1984); “Nucleic Acid Hybridization” Hames, B. D., and Higgins S. J., eds. (1985); “Transcription and Translation” Hames, B. D., and Higgins S. J., Eds. (1984); “Animal Cell Culture” Freshney, R. I., ed. (1986); “Immobilized Cells and Enzymes” IRL Press, (1986); “A Practical Guide to Molecular Cloning” Perbal, B., (1984) and “Methods in Enzymology” Vol. 1-317, Academic Press; “PCR Protocols: A Guide To Methods And Applications”, Academic Press. San Diego, Calif. (1990): Marshak et al., “Strategies for Protein Purification and Characterization—A Laboratory Course Manual” CSHL Press (1996); all of which are incorporated by reference as if fully set forth herein. Other general references are provided throughout this document. The procedures therein are believed to be well known in the art and are provided for the convenience of the reader. All the information contained therein is incorporated herein by reference.

### Example 1

### In Vitro Induction of Polyploidy in G. paniculata

The invention includes induced polyploidy in G. paniculata, the development of the genetic background referred to as M.S polyploidy. The manipulation of ploidy in plant tissue has been used to introduce fertility into hybrids and to produce plants with improved horticultural or agronomic traits (Kehr. A. E. 1996. Woody plant polyploidy. American Nurseryman. 183:38-47). In order to obtain seeds from the commercial variety Million stars (refer to as M.S), the present inventor employed an approach for treating cultured G. paniculata's explants with the antimitotic herbicide Oryzalin.

Materials and Methods

Internodal segments of cultivated G. paniculata (2 to 4 cm long) designated Million Stars™ were cultured in half strength Murashige and Skoog (MS) basal medium supplemented with 3% sucrose and 1% agar. Medium was sterilized by autoclave (121° C. for 20 min) and then supplemented with growth regulators for shoot multiplication (Gibberellin and benzylaminopurine). Oryzalin aqueous solution was added at final concentration of 0.05 mM to 0.5 mM, with 0.25% v/v Dimethyl sulfoxide (DMSO). Cultures were incubated at 23±2° C. under a 16 h photoperiod. Segments were re-cut after 2 weeks and placed into fresh half strength MS basal medium. After 2-3 weeks, regenerated shoots were cut and placed into MS basal solid medium for rooting, before subsequently being transferred to a greenhouse for acclimatization. Ploidy level of regenerated plantlets was evaluated by flow cytometry (Plant Cytometry Services—Laageinde 6 4016 CV Kapel Avezaath Buren, Netherlands) for all oryzalin treatments compared to un-treated varieties.

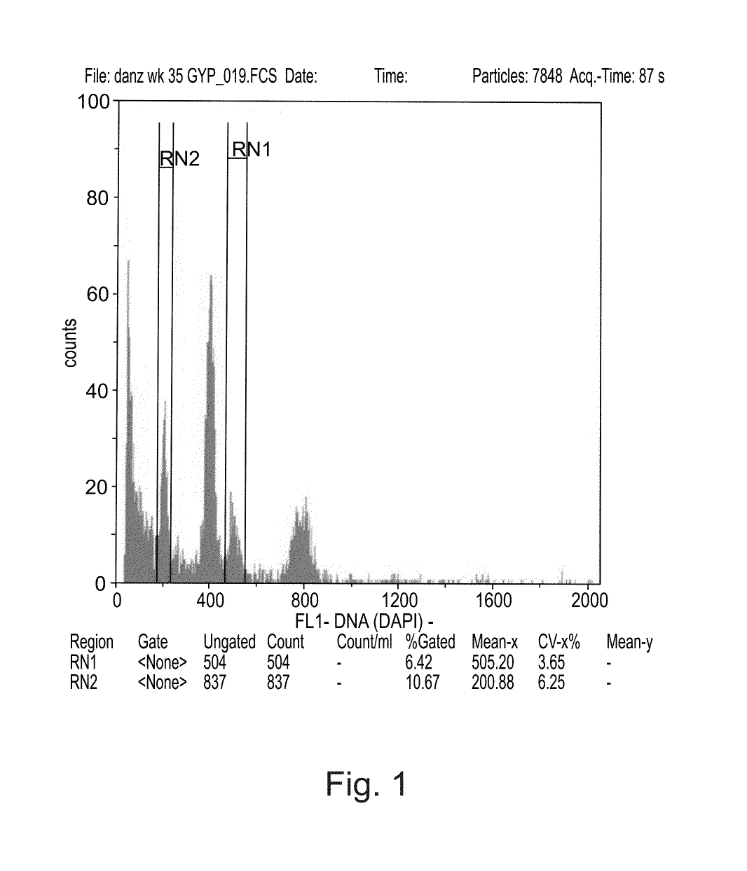

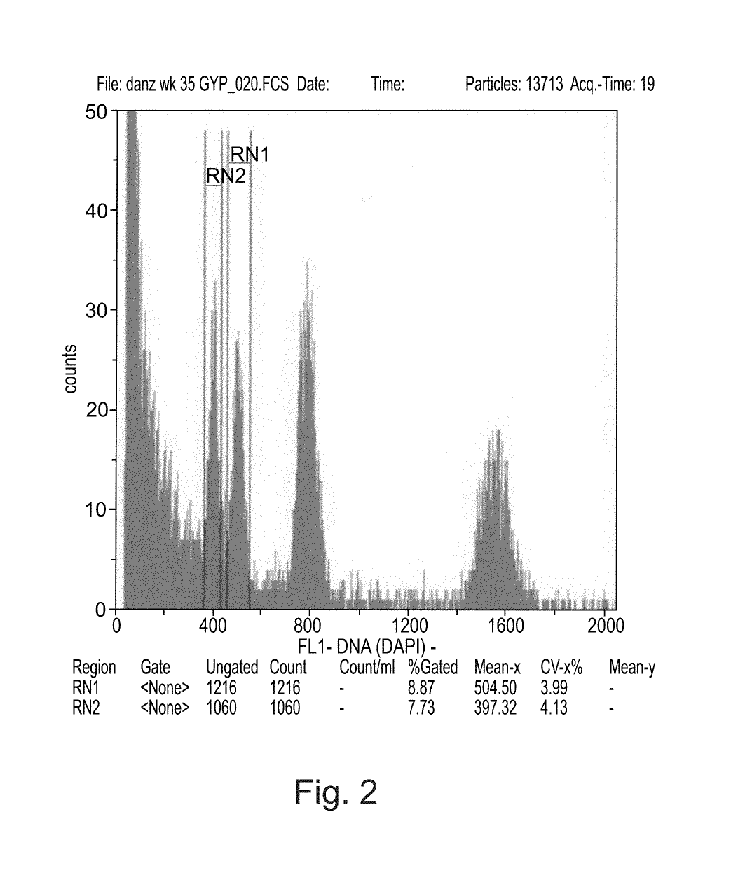

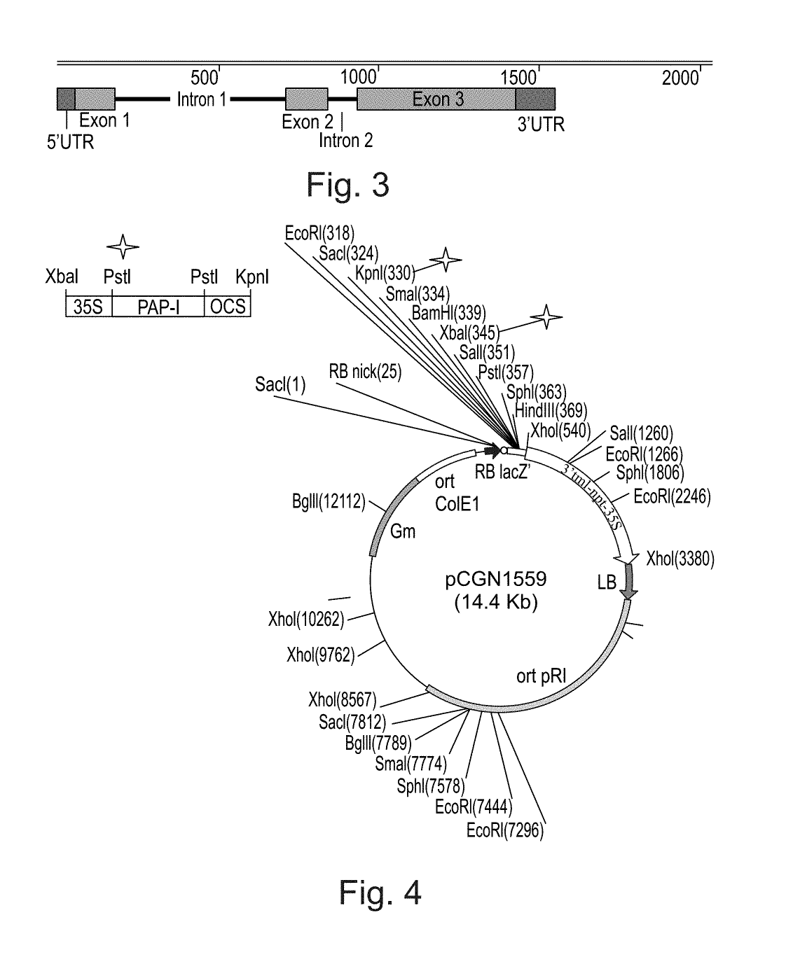

The outline of this approach is shown in Table IV and FIGS. 1 and 2. Oryzalin treatment of G. paniculata Million stars resulted in generation of the polyploid line designated as M.S polyploidy. In the DNA histograms, the peak of the internal standard (Vinca major) is marked as RN1.

The peak of the Gypsophila sample (representing the ploidy level) is marked with RN2.

TABLE IV
Flow cytometry results.

| | | | DNA | | |
|---|---|---|---|---|---|
| | | | ratio with | | |
| Species | Sample | Reference | int. st. * | Ploidy | Line |
| Gypsophila | GYP-19 | M.S | 0.40 | 2N | M.S |
| Gypsophila | GYP-20 | M.S | 0.79 | 4N | M.S |
| | | | | | polyploidy |

* Internal standard: Vinca major

Kehr, A E. (1996). Woody plant polyploidy. American Nurseryman. 183:38-47.
Murashige T & Skoog F, (1962). A revised medium for rapid growth and bioassays with tobacco tissue cultures. Physiol. Plant. 15:473-97.

### Example 2

### Genetic Backgrounds in G. paniculata

Different genetic backgrounds were used for the genetic transformation of Gypsophila, in order to obtain successful expression of PAP1. Seeds were collected from different open pollination fields between 2004 to 2009. Also, different Gypsophila species were used, Gypsophila scorzonerifolia, Gypsophila altissima, Gypsophila arborea, Gypsophila petraeus and Gypsophila arabica while screening for the genetic background in which a successful expression of PaP1 can be achieved. Within Gypsophila paniculata the genetic backgrounds used for transformation were Pinkolina, Miyabi and M.S Polyploid.

### Example 3

### Genetic Transformation of G. paniculata

Pap I (Production of Anthocyanin Pigment 1) is a Myb transcription factor known to regulate the production of phenylpropanoids, including anthocyanins (Borevitz J O, Xia Y, Blount J, Dixon R A, Lamb C. (2000). Activation tagging identifies a conserved MYB regulator of phenylpropanoid biosynthesis. The Plant Cell. 12, 2383-2393).

In the transformation construct pHAPAP (based on the binary vector pCGN1559) (FIG. 4), the pap I gene (SEQ ID NO: 1) is under transcriptional control of the 35S promoter from cauliflower mosaic virus (CaMV, SEQ ID NO: 4) and the transcription termination signal of the octopine synthase (ocs 3′) gene from Agrobacterium tumefaciens (SEQ ID NO: 3). The binary vector pHAPAP also contains the neomycin phosphotransferase gene (nptII) from Escherichia coli Tn5 under transcriptional control of the CaMV 35S promoter and tml 3′ terminator from Agrobacterium tumefaciens, which confers resistance to aminoglycoside antibiotics (Comai L. Moran P. Maslyar D. (1990). Novel and useful properties of a chimeric plant promoter combining CaMV 35S and MAS elements. Plant Mol Biol. 15(3). 373-381., McBride K E, and Summerfelt K R. (1990). Improved binary vectors for Agrobacterium mediated plant transformation. Plant Mol Biol 14, 269-276.).

Materials and Methods

Method of Transformation

Plant Material

Gypsophila (Gypsophila paniculata L.) seeds collected from different open pollination crossings that were conducted at Nir-Zvi. Israel, under natural field conditions year round. Seeds were isolated from the mother plant, cleaned and kept in a paper bags, under regular room temperature conditions. Each bag marked with a specific code which includes the genetic background of the seeds. All the seeds that were used for the transformation experiment were up to 6 month regular room temperature storage. Seeds were rinsed with 70% ethanol, sterilized for 10 min in 1.5% (w/v) sodium hypochlorite and rinsed three times in sterile water.

Media Composition and Tissue-Culture Conditions

Murashige and Skoog basal medium (MS; Murashige and Skoog 1962) with sucrose (30 g/l) and solidified with agar (8 g/l) (basic medium), was supplemented with growth regulators and antibiotics for co-cultivation with Agrobacterium (for transformation as described below), regeneration and selection of adventitious shoots, elongation and rooting of transgenic plants. All media were adjusted to pH 5.8 prior to autoclaving (121° C. for 20 min).

For germination, seeds were placed on basic medium containing 5 mg/gibberellic acid (GA) in a dark chamber, after ca. 5 days hypocotyls of the young embryos were isolated. For co-cultivation with Agrobacterium, the basic medium was supplemented with 0.1 mg/l α-naphthalene acetic acid (NAA), 0.5 mg/l 6-benzylaminopurine (BAP) and 100 μm acetosyringone (co-cultivation medium). Shoot regeneration and selection of transformants was performed on the basic medium supplemented with 0.1 mg/l NAA and 2 mg/l 1-phenyl-3(1,2,3-thiadiazol-5-yl)-urea (TDZ), medium referred to as “SR-T”.

Young leaves were separated from the regenerated tissue, and placed on new regeneration medium supplemented with 0.1 mg/l NAA and 1 mg/l 6-benzylaminopurine (BAP), medium referred to/designated as “SR-B”. Regeneration medium was also supplemented with 300 mg/l carbenicillin and, unless otherwise stated, 100 mg/l kanamycin. Elongation and rooting of transgenic shoots, following the second selection cycle, were performed on the basic medium containing 0.1 mg/NAA, 0.1 mg/l (GA), 200 mg/l carbenicillin and 100 mg/l kanamycin. All cultures were maintained in a growth room at 25±1° C. under a 16 h photoperiod using cool white light (60 μmol m-2 s-1), unless otherwise indicated.

Bacterial Strains

Agrobacterium tumefaciens strain AGLO (Lazo et al, 1991) carrying the binary vector pHAPAP (McBride K E and Summerfelt K R, 1990) from a single colony were grown at 28° C. for ca. 20 h in liquid LB medium (10 g/l bactotryptone, 5 g/l bacto-yeast extract, 5 g/l NaCl, 2 g/l glucose, pH 7.5) on a rotary shaker (250 rpm). The medium was supplemented with 100 μm acetosyringone, 50 mg/l rifampicin, and 25 mg/l gentamycin. Bacteria (AGLO/pHAPAP, O.D550=0.5) was harvested by centrifugation at 10,000×g for 2 min; the pellet was re-suspended in liquid co-cultivation medium (OD550=0.5 or 1.0), and the suspension was used for inoculation.

Transformation and Regeneration of Transgenic Plants

Hypocotyls explants were inoculated with bacterial suspension (AGLO/pHAPAP. O.D550=0.2). During co-cultivation and all consecutive steps, explants were cultured in an upright position. After 5 days of culture on the co-cultivation medium (3 days in the dark followed by 2 days in light), explants were transferred to SR-T medium for shoot regeneration and the first selection cycle. Explants were than cross-sectioned into two halves, and transferred to fresh SR-T medium. After ca. 3 additional weeks, clusters of regenerated adventitious shoots were excised from the primary stem explants. Leaves from all of the shoots of each independent cluster were pulled off and cultured on SR-B medium for adventitious shoot regeneration and selection of transgenes (second selection cycle). After 10 to 12 days, new adventitious shoots emerged from the leaf basal area. These shoots were transferred to elongation and then rooting media and evaluated as to their transgenic nature by PCR, as described in Moyal Ben Zvi, M., Zuker, A., Ovadis, M., Shklarman, E., Ben-Meir, H., Zenvirt, S. and Vainstein, A. (2008) Agrobacterium-mediated transformation of gypsophila (Gypsophila paniculata L.) Mol. Breeding 22:543-553. After hardening, transgenic plants were transferred to the greenhouse where they developed and flowered normally.

### Example 4

### Transgenic G. paniculata Varieties

Four transgenic lines were obtained by successful transformation with an expression of PAP1, the lines designated RP-1, RP-4 and RP-10. All of which were established from the same genetic background of the M.S polyploid, as described above.

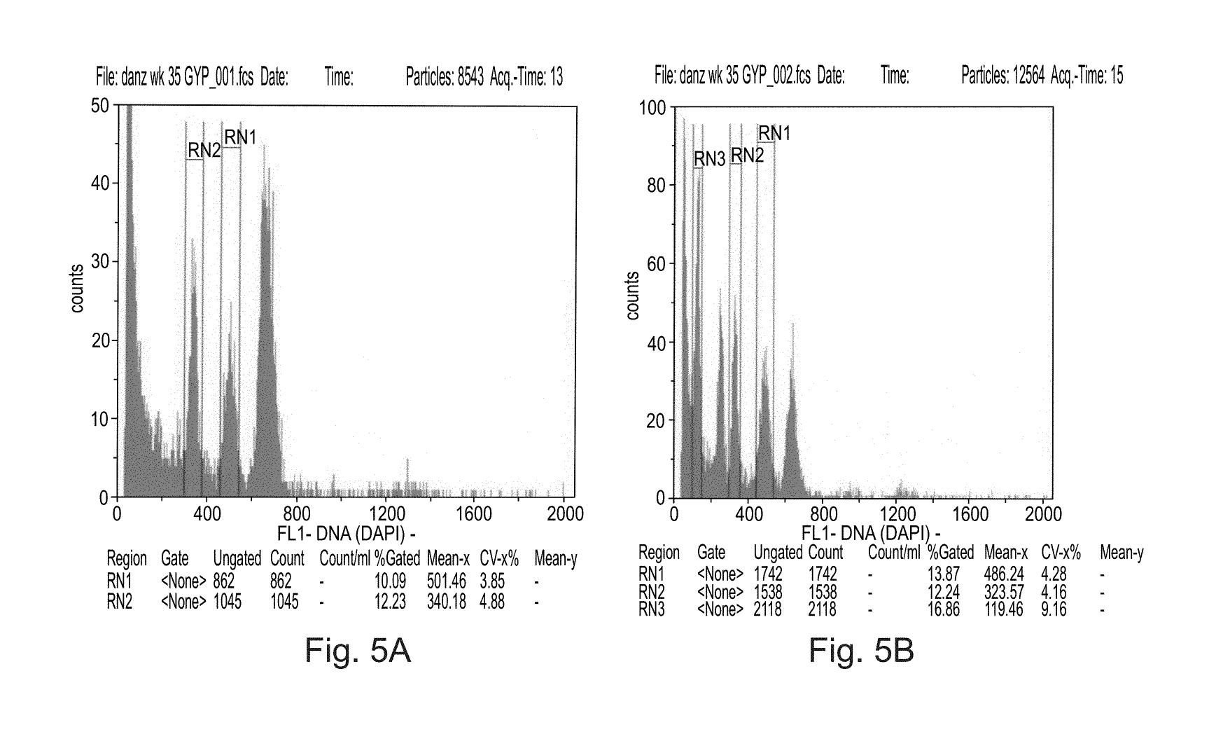

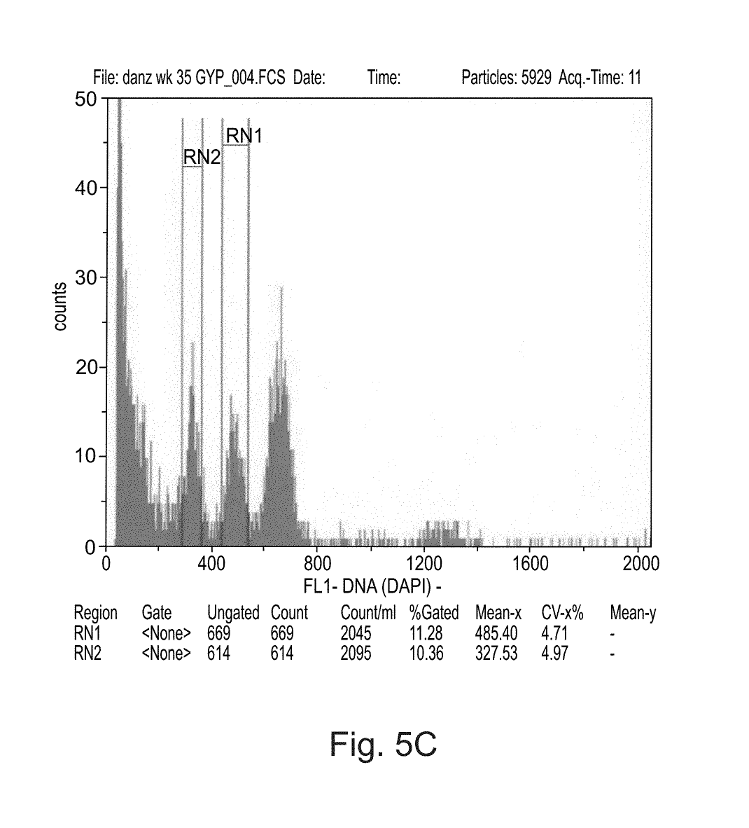

The breeding program was continued using those lines which are also characterized by ploidy level higher than 2×, as described in Table V, below, and FIGS. 5A-C.

TABLE V
Flow cytometry results.

| | | | DNA | | |
|---|---|---|---|---|---|
| | | | ratio with | | |
| Species | Sample | Reference | int. st. * | Ploidy | Line |
| Gypsophila | GYP-19 | M.S | 0.4 | 2x | M.S |
| Gypsophila | GYP-1 | M.S | 0.68 | High | RP-1 |
| | | | | polyploidy | |
| Gypsophila | GYP-2 | M.S | 0.66 | High | RP-4 |
| | | | | polyploidy | |
| Gypsophila | GYP-4 | M.S | 0.68 | High | RP-10 |
| | | | | polyploidy | |

* Internal standard: Vinca major

The breeding program continued with the above transgenic lines to create new varieties with a diverse range of phenotypes. The introduced pap I gene resulted in the accumulation of anthocyanin pigments in various plant tissues, including anthers, pistils, ovaries, petals, sepals, stems, and leaves. During subsequent crosses and selections, hybrids were produced with different pigment expression patterns and intensities in various flower organs such as green stem, green foliage (leaves), red flowers; dark stem, dark foliage (leaves), red flowers; and dark stem, dark foliage (leaves), white flowers. Examples for those phenotypes are described in Table VI, below.

TABLE VI
Phenotypes of different transgenic lines

| | | | | Anthers | | | | Plant | Stems | | | Flowering |
|---|---|---|---|---|---|---|---|---|---|---|---|---|
| | Color | Flower | Flower | Pigmen- | Sepals | Stem | Foliage | Height | Average | Stem | Leaf | Time |
| Line | Pattern | size | Type | tation | Pigmentation* | Pigmentation* | Pigmentation* | (cm) | Per Plant | Angels | Size | (weeks) |
| Explora | S | M | SD | Y | 1 | 1 | 1 | 100 | 7 | M | N | 9 |
| Dawn (59) | | | | | | | | | | | | |
| Explora | S | S | D | — | 0 | 1 | 1 | 110 | 8 | N-M | N | 10 |
| Sunrise | | | | | | | | | | | | |
| (505) | | | | | | | | | | | | |
| 450 | C | M | SD | Y | 1 | 1 | 1 | 75 | 6-8 | N-M | N | 9 |
| 272 | H | M | D | Y | 2 | 2 | 1 | 80 | 8 | W | M | 11 |
| 170 | H | M | SD | Y | 0 | 0 | 0 | 87 | 8 | W | W | 8 |
| 610 | S | M | DM | Y | 1 | 0 | 0 | 89 | 5-6 | W | M | 10 |
| 368 | S | M | SD | Y | 0 | 2 | 0 | 98 | 4 | W | M | 10 |
| 444 | S | M | SD | Y | 2 | 3 | 3 | 107 | 4 | W | W | 10 |
| 428 | S | S | SD | — | 2 | 3 | 2 | 110 | 5-6 | N | M | 10 |
| 611 | S | M | SD | Y | 0 | 0 | 0 | 120 | 5-6 | N | M | 10 |
| 604 | S | L | D | Y | 1 | 0 | 0 | 77 | 6 | M | N | 12 |
| 687 | S | M | SD | — | 0 | 0 | 0 | 100 | 7 | W | N-M | 11 |
| 649 | S | M | D | — | 0 | 0 | 0 | 110 | 8 | M | M | 12 |
| 606 | S | S | D | N | 2 | 1 | 1 | 90 | 8 | M | N | 11 |
| 706 | C | M | SD | — | 0 | 1 | 1 | 110 | 4-5 | N | M | 11 |
| 503 | S | XL | DM | — | 0 | 0 | 0 | 60 | 7 | N | M | 11 |
| RP-10 | H | S | SD | N | 2 | 3 | 1 | 110 | 10 | N | M | 10 |
| RP-4 | S | M | S | Y | 2 | 1 | 1 | 80 | 5 | W | W | 9 |
| RP-1 | S | S | SD | Y | 2 | 1 | 1 | 80 | 3-6 | W | N | 10 |

Color Pattern: H = homogenic, C = center, S = splash,
Flower size: S = Small, M = Medium, L = Large,
Flower type: S = Single, SM = Semi-Double, D = Double, DM = Double Multi-flowers,
Pigmentation*: 0 = none, 1 = low, 2 = medium, 3 = high,
Branch Height:, M = medium, T-Tall,
Stem Angels = narrow, W = wide, M = medium,
Anthers Pigmentation: Y = Yes, N = No.
Flower type: Single-a flower with one row of petals Semi-Double: a flower with more than one row of petals, and a clearly defined central which is visible. Double: a flower with a few rows of petals, and a central which is not visible. Double Multi-flowers: a flower with a few rows of petals, a central which is not visible and developing young buds are seen at the flower center.
Flower size: Small - smaller than 5 mm, Medium - between 6 to 9 mm, Large - between 9 to 12 mm. XL - larger than 12 mm.

The breeding program resulted in stable lines which represent new transgenic Gypsophila varieties that were selected for advancement and commercialization based on plant phenotype and agronomic performance, which include the level and pattern of pigment expression, as well as the fulfillment of commercial criteria such as plant architecture, flower morphology, agronomic performance, and economic yield.

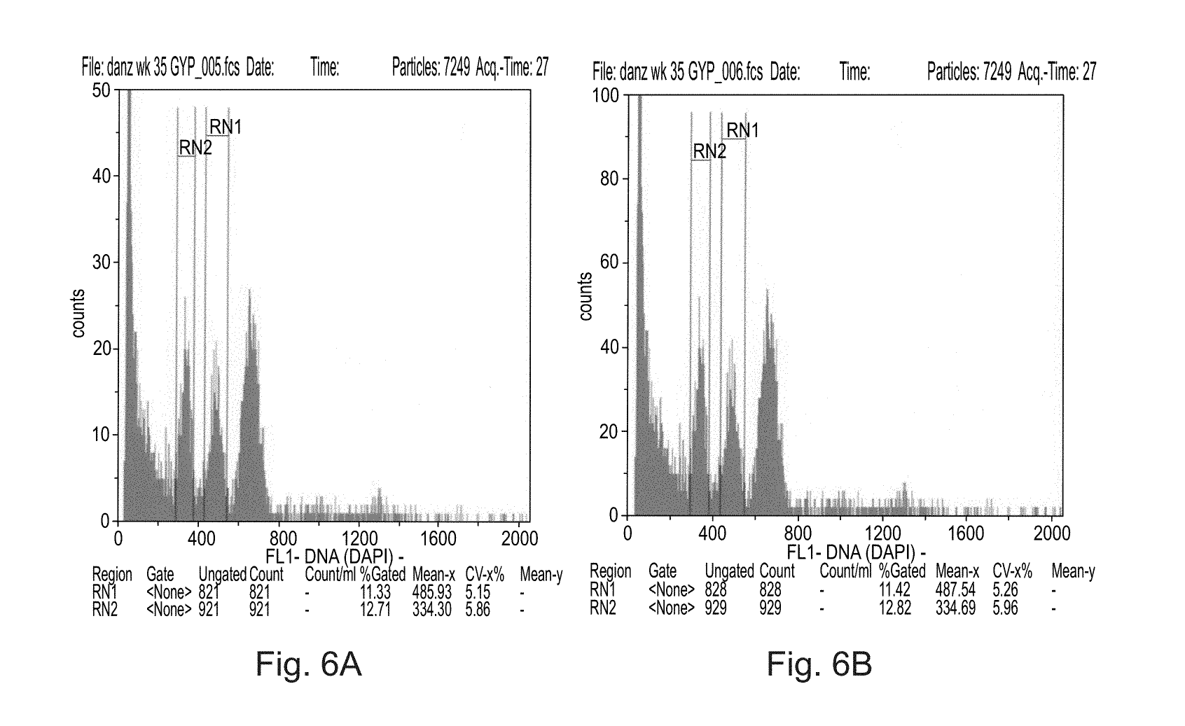

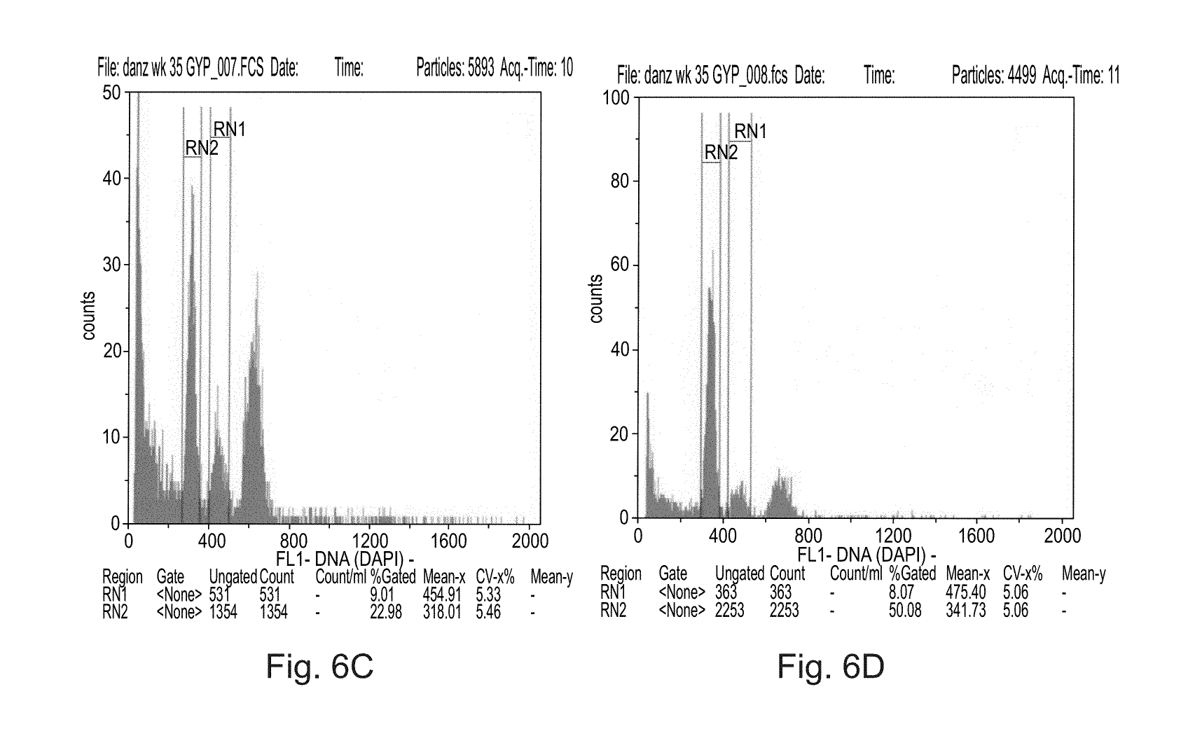

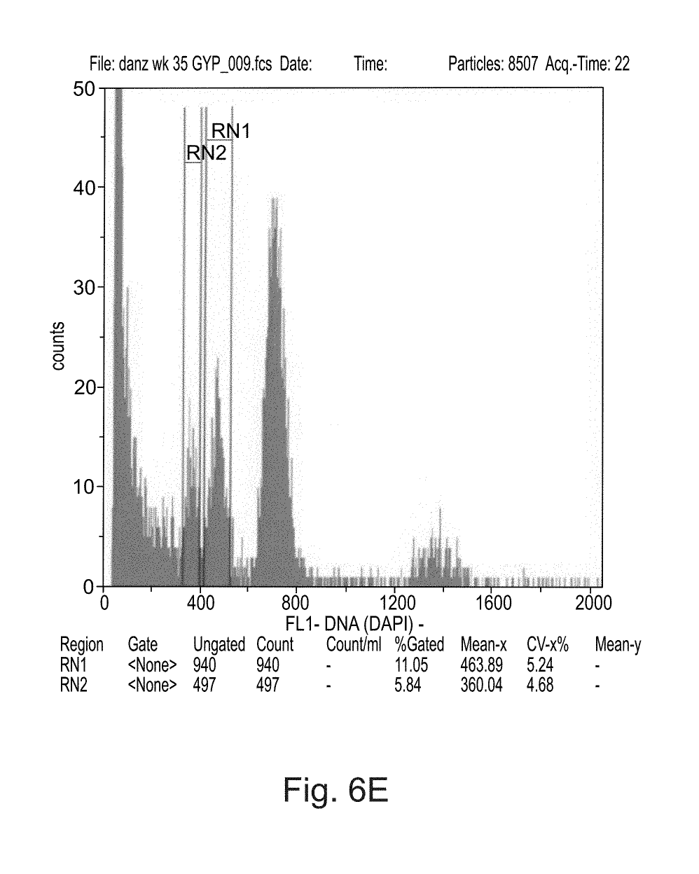

The DNA content of cells of some of the hybrid transgenic lines is described in Table VII below and FIGS. 6A-E.

TABLE VII
Flow cytometry results.

| | | | DNA | | |
|---|---|---|---|---|---|
| | | | ratio with | | |
| Species | Sample | Reference | int. st. * | Ploidy | Line |
| Gypsophila | GYP-19 | M.S | 0.4 | 2x | M.S |
| Gypsophila | GYP-5 | M.S | 0.69 | High | TG-59 |
| | | | | polyploidy | |
| Gypsophila | GYP-6 | M.S | 0.69 | High | TG-505 |
| | | | | polyploidy | |
| Gypsophila | GYP-7 | M.S | 0.7 | High | TG-365 |
| | | | | polyploidy | |
| Gypsophila | GYP-8 | M.S | 0.72 | High | TG-450 |
| | | | | polyploidy | |
| Gypsophila | GYP-9 | M.S | 0.78 | High | TG-272 |
| | | | | polyploidy | |

* Internal standard: Vinca major

### Example 5

### Pigment Analysis in Transgenic G. paniculata Varieties

Flowers from a number of transgenic hybrid Gypsophila varieties generated according to the present teachings were subjected to pigment analysis.

Materials and Methods

Plant Material

Flower for anthocyanins extraction were taken from flowering plant in Israel during the summer (August), those plants were planted in greenhouse on March that year. The plants were grown as follow: planting density was 6 plants/m² bed, the plants were pinched 4 weeks after planting and sprayed with 400 ppm of gibberellic acid 6 weeks after planting.

Anthocyanins Determination by UPLC-QTOF-MS

100 mg of frozen fine powder of Gypsophila were extracted by 70% methanol+2% formic acid at a ratio of 1:3 w/v (tissue:extraction solution) followed by min in bath sonicator and centrifugation for 10 min at 13,000 rpm. The supernatant was filtered through 0.22 μn PTFE membrane filter (Acrodisc® CR 13 mm; PALL) before injection to UPLC-QTOF-MS instrument.

Mass spectral analysis of anthocyanins was carried out with an UPLC-QTOF instrument (Waters HDMS Synapt G2-S), with the UPLC column connected online to a PDA detector (190-800 nm) and then to the MS detector. Separation of metabolites was performed by gradient elution (acetonitrile-water, containing 3% formic acid) on a 100×2.1 mm i.d., 1.7 μm UPLC BEH C18 column (Waters Acquity) at a flow rate of 0.1 ml/min. The linear gradient was as follows: 100 to 65% phase A over 25 min, 65 to 0% phase A over 5 min, held at 100% phase B for further 1 min; and then returned to the initial conditions (100% phase A) in 0.2 min and conditioning at 100% phase A for 4 min. Injection volume was 0.5 μl. Masses of the eluted compounds (m/z range from 50 to 1200 Da) were detected with a QTOF-MS equipped with an ESI source performed in positive mode using MSE mode. The collision energy was set to 4 eV for low-energy function and 15-50 eV ramp for high-energy function. A mixture of 15 standard compounds, injected after each 10 samples, was used for QC. Compounds were putatively identified by comparison of the observed UV spectra, MS fragments and determined elemental composition with those found in the literature.

Results

The results of pigment analysis are summarized in Table VIII below and shown in FIG. 7.

Transgenic gypsophila varieties show increase in the levels of anthocyanins compare to non-transgenic white flowering Gypsophila (M.S) and light pink Gypsoiphila My Pink™ (M.P).

Pigment analysis showed a range of anthocyanins amounts in the transgenic varieties, ratios of cyanidin malylglucoside in variety 170 were 1.7 and 2.4 fold higher compare to varieties 59 and 100, respectively. Ratios of cyanidin hexose in variety 170 were 0.9 and 1.7 fold higher compare to varieties 59 and 100, respectively. Ratios of Peonidin coumaroyl-pentose in variety 170 were 54.7 and 2.2 fold higher compare to varieties 59 and 100, respectively.

Pigment analysis showed that cyanidin hexose and the peonidin derivatives, that were identified in the transgenic varieties and in M.P, were not identified in M.S. Ratios of cyanidin malylglucoside in variety 170 were 116 and 7545 times higher compare to varieties M.P and M.S, respectively. Ratios of cyanidine pentose deoxyhexose in variety 170 were 98 and 75 times higher compare to varieties M.P and M.S. respectively.

TABLE VIII
Anthocyanins analysis of G. paniculata varieties

| | | | Peonidin | Cyanidine |
|---|---|---|---|---|
| | Cyanidin | | coumaroyl- | pentose |
| variety | malylglucoside | Cyanidin hexose | pentose | deoxyhexose |
| 170 | 10340753.67 ± 289705.35 | 979290.9 ± 45469.88 | 500120.63 ± 8775.34 | 128284.37 ± 3589.3 |
| 59 | 6003251.75 ± 179499.75 | 1099718.94 ± 26500.19 | 9147.66 ± 201.69 | 208327.81 ± 6166.19 |
| 100 | 4229912.25 ± 119851 | 560080.56 ± 31290.5 | 223039.8 ± 7325.27 | 65140.04 ± 2306.15 |
| MP | 88785.17 ± 3274.75 | 15138.62 ± 1170.39 | 1827.78 ± 48.43 | 1298.79 ± 118.82 |
| MS | 1370.42 ± 65.81 | 0 ± 0 | 0 ± 0 | 1708.56 ± 27.06 |
| 170 vs | 1.7 | 0.9 | 54.7 | 0.6 |
| 59 | | | | |
| 170 vs | 2.4 | 1.7 | 2.2 | 2.0 |
| 100 | | | | |
| 170 vs | 116.5 | 64.7 | 273.6 | 98.8 |
| Mp | | | | |
| 170 vs | 7545.7 | — | — | 75.1 |
| MS | | | | |

| | Unknown | Unknown | Unknown | Cyanidin |
|---|---|---|---|---|
| | cyanidin | peonidin | pelargonidin | coumaroyl- |
| variety | derivative | derivative | derivative | deoxyhexose |
| 170 | 20798.38 ± 534.99 | 2016.7 ± 145.84 | 8560.82 ± 418.06 | 0 ± 0 |
| 59 | 48954.66 ± 358.87 | 562.99 ± 32.13 | 1255.43 ± 184.57 | 2878.24 ± 57.18 |
| 100 | 40815.2 ± 1401.24 | 6122.94 ± 230.08 | 1416.28 ± 105.19 | 0 ± 0 |
| MP | 845.1 ± 28.07 | 53.67 ± 39.89 | 0 ± 0 | 0 ± 0 |
| MS | 21.21 ± 21.21 | 0 ± 0 | 0 ± 0 | 0 ± 0 |
| 170 vs | 0.4 | 3.6 | 6.8 | — |
| 59 | | | | |
| 170 vs | 0.5 | 0.3 | 6.0 | — |
| 100 | | | | |
| 170 vs | 24.6 | 37.6 | — | — |
| Mp | | | | |
| 170 vs | 980.7 | — | — | — |
| MS | | | | |

### Example 6

### Molecular Characterisation of Gypsophila Transgenic Lines

Materials and Methods

DNA PCR

DNA PCR analysis of transgenic lines was carried out with PAP1 primers using methods outlined in Moyal-Ben Zvi et al. (2008b).

The following primers were used:

PAP F438
SEQ ID NO: 5
TTC CTA CAA CAC CGG CAC TAA/
PAP R728
SEQ ID NO: 6
TTT CTG TTG TCG TCG CTT CA/

Southern Analysis

To confirm stable integration into the genome, in PCR-positive PAP1 gypsophila plants. Hind III-digested genomic DNA of nine gypsophila plants was used for Southern blotting analysis, using a probe for the PAP1 gene.

| Probe | Primer 5′ → 3/SEQ ID NO: |
|---|---|
| PAP1-F3870 | ACGCCCATTCCTACAACAC/7 |
| PAP1-R4224 | TCTCTCCATCGAAAAGACTCC/8 |

gDNA was isolated from 10 tissue samples of the following transgenic lines: 1, 4, 7, 10, 59, 272, 365, 450, 505 (M.S. was used as a negative control) using the NucleoSpin Plant II Maxi Kit (Macherey-Nagel) according to manufacturer's instruction. gDNA quality was checked on gel and concentration was determined by Qubit analysis. Approximately 5 μg of each gDNA and ˜25 pg of the probe plasmid DNA were digested with HindII and loaded onto 350 ml, 0.65% TBE agarose gel with 1 Kb+Marker. The gel was electrophoresed at 60V for 17.5 hours. Gels were stained, photographed and then treated with 0.5 M NaOH+1.5 M NaCl two times for 25 minutes each followed by treatment with 0.5 M tris. pH8.0+1.5 M NaCl twice for 25 minutes each. The gel was transferred to nylon membranes. Nytran Supercharge (Whatman), according to manufacturer's instructions using a ‘TurboBlotter’ and 10×SSC overnight. The membrane was UV linked and air-dried. The filter was prehybridized using 6×SSC. 5×Denhardt's solution and 0.5% SDS at 68° C. for 7 hours. The filter was hybridized using their respective random primed probe template DNA, PAP1. Specific activities of greater than 1×10⁹ dpm/ug for the probes were achieved. For hybridization, the probe concentration was ˜3.5×10⁶ dpm/ml in the HYBE buffer (same as the prehybridisation buffer). The hybridization was carried out at 68° C. for 16.5 hours. The filter was washed in 2×SSC+0.1% SDS at 68° C. with three buffer changes over a period of 60 minutes. The filter was autoradiographed for 4 days with an intensifier screen at −80° C.

Results

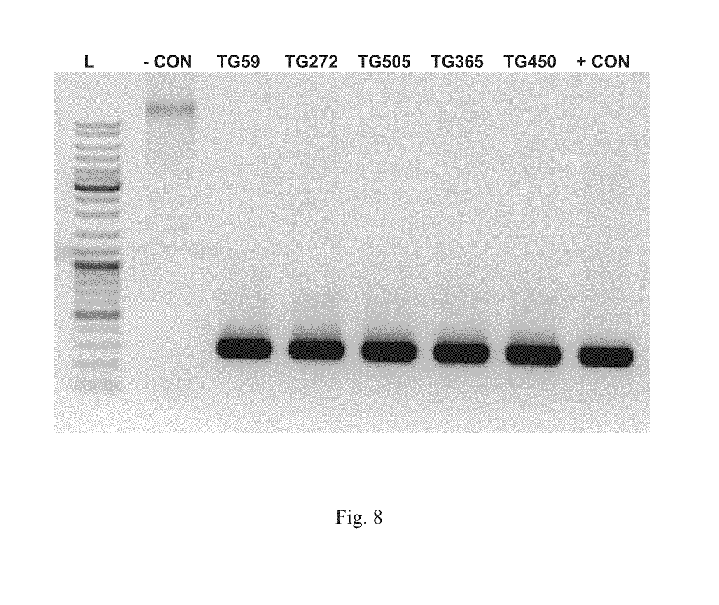

DNA PCR analysis of transgenic lines carried out with PAP1 primers confirmed the presence of the gene in the transgenic plants (FIG. 8).

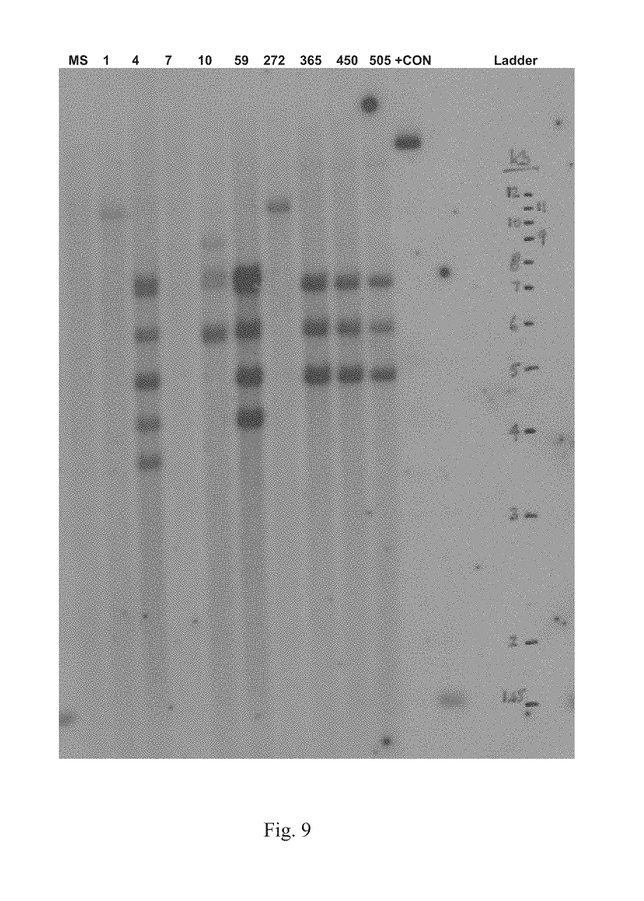

Southern analysis was done to show stable integration of PAP1 in the genome of the transgenic lines. Results are shown in FIG. 9. The results show that at least 1-5 hybridization bands appeared in eight transgenic lines, but not in the non-transformed plant Million Stars (lane M.S) or in the other negative control plant referred to as RP-7, indicating that the PAP1 gene was integrated into the genomes of the tested plants.

Hind III cuts only at one site in the T-DNA region (FIG. 9), therefore the number of bands produced in Southern blot hybridization should reflect the number of integrated sites of the transgene in each plant. Southern blot results suggest the presence of at least three integration sites in the genomes of plants TG505 and TG450 and at least four copies in the genome of plant 59. TG505, TG59 and TG450 were obtained after breeding from the same transgenic event (RP-4) and hence have similar integration sites.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

1. A Gypsophila paniculata plant comprising an exogenous nucleic acid sequence encoding PAP1.

2. A Gypsophila paniculata plant comprising a flower producing a non-thermally induced red, pink, purple or green pigmentation or a combination of same.

3. The plant of claim 2, comprising an exogenous nucleic acid sequence encoding PAP1.

4. The plant of claim 1, wherein said exogenous nucleic acid sequence encoding PAP1 is stably integrated in a genome of the plant.

5. The plant of claim 4, wherein said exogenous nucleic acid sequence encoding PAP1 is comprised in multiple copies within said genome.

6. The claim 1, being a polyploid.

7. (canceled)

8. The plant of claim 1, having a pedigree which includes a Million Stars™ or Million Stars™ polyploid.

9. (canceled)

10. The plant of claim 1, wherein said PAP1 comprises an amino acid sequence as set forth in SEQ ID NO: 2.

11. The plant of claim 1, having flower petals that contain cyanidin as the major anthocyanin or wherein said flower petals further comprise peonidin, and pelargonidin derivates.

12. (canceled)

13. The plant of claim 11, wherein said cyanidin comprises cyanidin malylglucoside and cyanidin hexose.

14. The plant of claim 13, wherein said cyanidin malylglucoside and said cyanidin hexose are about 80-90% and 10-20% respectively, of total anthocyanin content of said flower petals, as assayed by UPLC-QTOF-MS.

15. The plant of claim 1, having flower petals that contain at least one of:
(i) at least 10, 20, 50, 100 or 150 fold increase in cyanidin malylglucoside than that found in My Pink™ at the same developmental stage and assay conditions;
(ii) at least 1,000, 2,000, 3000, 5,000, 8,000 or 10,000 fold increase in cyanidin malylglucoside than that found in Million Stars™ at the same developmental stage and assay conditions;
(iii) at least 10, 20, 50, 100 or 150 fold increase in cyanidin hexose than that found in My Pink™ at the same developmental stage and assay conditions;
(iv) at least 100, 200, 300, 500 or 1000 fold increase in peonidin coumaroyl pentose than that found in My Pink™ at the same developmental stage and assay conditions;
(v) at least 50, 100, 200 or 500 fold increase in cyanidin pentose deoxyhexose than that found in My Pink™ at the same developmental stage and assay conditions.

16-18. (canceled)

19. A flower of the plant of claim 1.

20. The flower of claim 19 being a cut-flower.

21. (canceled)

22. A seed of the plant of claim 1.

23. (canceled)

24. A cutting of the plant of claim 1.

25. (canceled)

26. The plant or plant part of claim 1, wherein the plant is a hybrid plant.

27. The plant or plant part of claim 1, wherein the plant is an inbred plant.

28. The plant or plant part of claim 1, wherein said hybrid plant or inbred plant is polyploid.

29. A method of producing a Gypsophila plant, the method comprising:
(a) crossing the plant or plant part of claim 1 with another Gypsophila plant;
(b) recovering seeds following said crossing;
(c) planting said seeds and growing said seed into plants; and
(d) selecting a hybrid plant.

30. The method of claim 29, wherein said selecting is according to pigmentation.

31. A hybrid plant or part thereof produced according to the method of claim 29.

32. (canceled)

33. A method of developing the plant of claim 1, the method comprising:
(a) crossing a Gypsohila paniculata plant with another Gypsohila paniculata plant so as to obtain hybrid seeds; and
(b) growing plants of said hybrid seeds; and
(c) selecting a plant of said plants that exhibits a flower producing a non-thermally induced red, pink, purple or green pigmentation or a combination of same.

34. A method of producing a transgenic Gypsophila paniculata comprising introducing into a Gypsophila paniculata plant a nucleic acid sequence encoding PAP1 operably linked to a cis-acting regulatory element active in a plant cell, thereby producing a transgenic Gypsophila paniculata, and optionally wherein the method further comprises subjecting the Gypsophila paniculata plant to polyploidization protocol.

35. (canceled)

36. The method of claim 34, wherein said Gypsophila paniculata plant has a Million Starts™ polyploid genetic background.

37. (canceled)
